(19) United States
(12) Patent Application Publication
(10) Pub. No.: US 2007/0099826 A1
(43) Pub. Date: May 3, 2007

(54) Treatment of diseases associated with the egr-1 enhancer element

(76) Inventors: Norman Wong, Alberta (CA); Joseph Tucker, Alberta (CA); David R. McCaffrey, Alberta (CA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/575,406

(22) PCT Filed: Oct. 8, 2004
(86) PCT No.: PCT/CA04/01818

§ 371 Date: Nov. 1, 2006

(30) Foreign Application Priority Data
Oct. 10, 2003 (US) 60510669
Oct. 10, 2003 (US) 60510342
Jan. 22, 2004 (US) 10762796
Mar. 24, 2004 (US) 10807800

(51) Int. Cl. A61K 38/18; A61K 31/7048; A61K 31/655; A61K 31/353; A61K 31/21; A61K 31/135; A61K 31/05

(52) U.S. Cl. 514/12; 514/150; 514/731; 514/646; 514/706; 514/509; 514/27; 514/456

(57) ABSTRACT

Compounds and methods are provided for treating patients suffering from health condition associated with an expression state of a gene such as fertility disorders, cancer, proliferative diseases, vascular diseases, wounds requiring therapeutic intervention, inflammation, and pulmonary disorders by administering to said patient a compound capable of modulating egr-1 and/or an egr-1 response element consensus sequence thereby altering the expression state of said gene. Also described are new methods for screening compounds to identify effectors of egr-1 and/or egr-1 consensus sequence elements and methods for treating patients by administering such effectors to modulate egr-1 and/or egr-1 consensus sequences to thereby modify expression of genes associated therewith to in turn treat diseases or other physiological conditions associated with such gene expression.

### FIELD OF INVENTION

The present invention describes a method for screening compounds for regulating expression of APO A1 protein and modulating the activity of egr-1 and/or egr-1 consensus sequence elements for influencing expression of associated genes to thereby effect disease treatment.

### BACKGROUND OF INVENTION

Cardiovascular disease is a general term used to identify a group of disorders of the heart and blood vessels including hypertension, coronary heart disease, cerebrovascular disease, peripheral vascular disease, head failure, rheumatic heart disease, congenital heart disease and cardiomyopathies. The leading cause of cardiovascular disease is atherosclerosis, the build up of lipid deposits on arterial walls. Elevated levels of cholesterol in the blood are highly correlated to the risk of developing atherosclerosis, and thus significant medical research has been devoted to the development of therapies that decrease blood cholesterol.

Atherosclerosis is associated with endothelial dysfunction, a disorder wherein normal function of the vasculature lining is impaired, which contributes to the pathogenesis of atherosclerosis in addition to being a prominent risk factor for numerous other cardiovascular disorders such as angina, myocardial infarction and cerebrovascular disease. Hallmarks of endothelial dysfunction include increased oxidative vascular stress and vasoconstriction, as well as elevated levels of cholesterol in the blood, which all promote one another to accelerate the development of cardiovascular disease. In order to most successfully disrupt the development of disease, improved therapeutic strategies against-the multiple causal risk factors of cardiovascular disease are needed.

Resveratrol (trans-3,5,4′-trihydroxystilbene) is a natural polyphenol found in certain plants and berries including red grapes, raspberries, mulberries, peanuts and some other plants. It has been suggested that resveratrol, its metabolites and related polyphenols present in red wine may underlie an epidemiologic observation termed the “French Paradox”. This paradox relates to the finding of a low incidence of cardiovascular disease (CVD) in the French population despite the consumption of a diet containing a high content of saturated fat comparable to that in the North American population. The content of saturated fat in the North American diet is a major contributor to the incidence of ischemic heart disease. In France, however, a comparable diet is associated with an incidence of ischemic heart disease equal to ⅓ of that in the North American population. It has been speculated that resveratrol may contribute to the paradox comes from its potential role as an antioxidant and additionally, as yet unknown mechanism(s) of action. Resveratrol and related compounds are found in abundance in nature and one of the best known sources are the skins of red grapes, which can contain 50-100 μg per gram (Jang, M. et al. Science 275:218 (1997)) of skin. Resveratrol is found in many red wines and may also be obtained in commercial preparations.

In part, the actions of resveratrol may arise, from its suspected antioxidant properties that inhibit lipid peroxidation of low-density lipoprotein (LDL) particles and thus prevent the cytotoxicity of oxidized LDL. Increased abundance of oxidized LDL is a risk factor for developing CVD (Frankel, E. N. et al. Lancet 341:1103 (1993); Chanvitayapongs, S. et al. Neuroreport 8:1499 (1997)). Platelet aggregation in the pathogenesis of CVD occurs at early and late stages of the disease including the final insult of arterial thrombosis. This is usually the terminal event leading to ischemia or myocardial infarction. Thus the ability of resveratrol to inhibit this platelet activity is thought to possibly help in both prevention of atherosclerosis (Rotondo, S. et al. Brit J Pharmacol 123:1691 (1998); Soleas, G. J. et al. Clin Biochem 30:91 (1997)) and the final insult. These effects of resveratrol may comprise, in part, the cardioprotective effects of moderate amounts of red wine consumption.

Cholesterol Metabolism

Due to its insolubility, cholesterol is transported in the blood by complexes of lipid and protein termed lipoproteins. Low density lipoproteins (LDL) are believed to be responsible for the delivery of cholesterol from the liver to other tissues in the body, and have thus become popularly referred to as “bad cholesterol”. LDL particles are converted from intermediate density lipoproteins (IDL) which were themselves created by the removal of triglycerides from very low density lipoproteins (VLDL). VLDL are synthesized out of triglycerides and sever apolipoproteins in the liver, where they are then secreted directly into the bloodstream.

High density lipoproteins (HDL) are thought to be the major carrier molecules that transport cholesterol from extrahepatic tissues to the liver where it is catabolized and then eliminated in a process termed reverse cholesterol transport (RCT), thereby earning HDL the moniker of the “good cholesterol”. In the elimination process that occurs in the liver, cholesterol is converted to bile acids and then excreted out of the body.

Current Treatments for Hyperlipidemias

Currently approved cholesterol lowering drugs provide therapeutic benefit by attacking the normal cholesterol metabolic pathways at a number of different points. Bile acid binding resins, such as cholestyramine, adsorb to bile acids and are excreted out of the body, resulting in an increased conversion of cholesterol to bile acids, consequently lowering blood cholesterol. Resins only lower serum cholesterol a maximum of 20%, cause gastrointestinal side effects and can not be given concomitantly with other medications as the resins will bind to and cause the excretion of such other drugs.

Niacin inhibits lipoprotein synthesis and decreases production of VLDL particles, which are needed to make LDL. When administered at the high concentrations necessary to increase HDL levels, serious side effects such as flushing occur.

Fibrates, such as clofibrate and fenofibrate, are believed to activate transcription factors belonging to the peroxisome pro liferator-activated receptor (PPAR) family of nuclear hormone receptors. These transcription factors up-regulate genes involved in the production of HDL and down-regulate genes involved in the production of LDL. Fibrates are used to treat hyperlipidomias because they reduce serum triglycerides by lowering the VLDL fraction. However, they have not been approved in the United States as hypercholesterolemia therapeutics, due to the heterogeneous nature of the lipid response in patients, and the lack of efficacy observed in patients with established coronary heart disease. As well, the use of fibrates is associated with serious side effects, such as gastrointestinal cancer, gallbladder disease and an increased incidence in non-coronary mortality.

Statins, also known as HMG CoA reductase inhibitors, decrease VLDL, LDL and IDL cholesterol by blocking the rate-limiting enzyme in hepatic cholesterol synthesis, Statins increase HDL levels only marginally, and numerous liver and kidney dysfunction side effects have been associated with the use of these drugs.

Ezetimibe is the first approved drug in a new class of cardiovascular therapeutics, which functions by inhibiting cholesterol uptake in the intestine. Ezetimibe lowers LDL but does not appreciably increase HDL levels, and does not address the cholesterol which is synthesized in the body nor the cholesterol circulating in the bloodstream or present in atherosclerotic plaques. Other compounds that have also bean discovered to affect cholesterol absorption include the bile-acid binding agent cholestyramine and the phytosterols.

Despite the development of these therapeutic approaches, little has been achieved to increase the blood levels of HDL, and all of the drugs currently approved are limited in their therapeutic effectiveness by side effects and efficacy. Consequently, there is a need for improved therapeutic approaches to safely elevate HDL and thus increase he rate of reverse cholesterol transport to reduce blood levels of cholesterol.

Endothelial Dysfunction and Atherosclerosis

Impaired endothelial function occurs early in the genesis of atherosclerosis, and in fact is detectable before lipid deposits. Endothelial dysfunction is symptomatically characterized by vasoconstriction and leads to hypertension, which is a well known risk factor for other cardiovascular disorders such as stroke and myocardial infarction.

Research has causally linked the diminished endothelial function in atherosclerosis patients to reduced bioavailability of nitric oxide (NO), a signaling molecule that induces vasodilation.

Decreased bioavailability of NO also activates other mechanisms that play a role in the pathogenesis of atherosclerosis. For instance NO is well known to inhibit platelet aggregation, a necessary step in the development of the lipid plaques that characterize atherosclerosis. As well, NO is an important endogenous mediator that inhibits leukocyte adhesion, which is a major step in the development of atherosclerosis and is probably the result of increased vascular oxidative stress in hyperlipidemic patients. Adherent leukocytes further increase oxidant stress by releasing large amounts of reactive oxygen species.

Increased vascular oxidative stress and hypercholesterolemia have individually been identified as contributors to the cause of reduced NO bioavailability. Increased oxidation also leads to free-radical mediated lipid peroxidation, another inducer of atherosclerosic lesion formation. In summary, it would appear that a positive feedback loop exists wherein these three major factors, hypercholesterolemia, vascular oxidative stress and reduced bioavailability of NO, each increase the extent and pathological severity of the others.

Resveratrol as an Anti-Oxidant and Pro-Apolipoprotein A1 Agent

The mechanism by which resveratrol reduces the incidence of cardiovascular disease remain a topic of considerable debate, with several competing hypotheses. Resveratrol has been demonstrated to be a potent anti-oxidant, which is suggested to result in lower levels of peroxidation of LDL particles, and subsequently to inhibit atherogenesis. Resveratrol has also been implicated as an inhibitor of leukocyte adhesion and platelet aggregation. In addition, resveratrol is being investigated as a potential anti-cancer therapeutic due to its described capability of modulating the activity levels of p21 and p53.

Resveratrol has been identified as an anti-inflammatory agent, with proposed mechanisms including the inhibition of the cyclooxygenase-1 enzyme (U.S. Pat. No. 6,541,045; Jayatilake, G. S. et al. J Nat Prod 56:1805 (1993); U.S. Pat. No. 6,414,037) and protein kinase inhibition (US Patent Application 0030171429). Consequently, resveratrol may have the potential to be employed therapeutically to treat arthritic disorders, asthmatic disorders, psonatic disorders, gastrointestinal disorders, ophthalmic disorders, pulmonary inflammatory disorders, cancer, as an analgesio, as an anti-pyretic, or for the treatment of inflammation that is associated with vascular diseases, central nervous system disorders and bacterial, fungal and viral infections.

Resveratrol was recently described as a sirtuin-activating compound, and was suggested to increase longevity through a direct interaction with SirTl, leading to down-regulation of p53. Resveratrol is also known to antagonize the aryl hydrocarbon receptor and agonize the estrogen receptor, and has been described to mediate activity through activation of the ERK 1/2 pathway and through increasing the activity of the transcription factor egr-1.

Most recently, resveratrol has been found to increase the transcription of apolipoprotein A1, putatively mediated through Site S, a nucleotide sequence in the promoter region of the ApoA-1 gene (Taylor et al. J Mol Endocrin 25:207(2000)).

### SUMMARY OF INVENTION

It is an object of the present invention of the present invention to provide an increased understanding of the mechanisms of action to resveratrol and to provide a basis for the development of resveratrol analogues that have similar beneficial actions.

It is a further object of the present invention to provide a molecular target for further drug development aimed at increasing APO A1 and/or HDL levels.

It is a further object of the present invention to provide novel compounds that are capable of increasing egr-1 promoter activity.

In accordance with the various aspects and principles of the present invention there are provided new tools and reagents for assaying and identifying compounds which can to increase HDL levels by promoting APO A1 gene expression. Various regions related to the APO A1 gene and specifically within the relevant promoter region have been identified that appear to be important for controlling gene activity. Polyphenol compounds such as resveratrol have been discovered to enhance activity of the gene. Cell lines have been discovered and created which are useful as screening tools for identifying other such compounds including mimetics and analogs of resveratrol for upregulating APO A1 gene expression. Similarly, such tools can be advantageously employed to screen synthetic compounds or neutraceuticals for identifying those compounds capable of providing similar benefit on APO A1 expression.

One aspect of the present invention provides methods for increasing HDL/APO A1 levels in plasma in an individual by administering therapeutically effective amount of an activating agent for selectively promoting APO A1 expression in intestinal and liver cells. Such activating agent acts upon the DNA within the intestinal cells, specifically at a DNA motif spanning −190 to −170 of the gene. It has been discovered that resveratrol or analogs thereof can act as such activating agents. Most preferred embodiments of such compounds will also comprise a pharmaceutically acceptable carrier such as a buffer, or other vehicle well known in the art.

A further aspect of the present invention provides for novel methods of promoting APO A1 expression, particularly in intestinal cells.

A further aspect of the present invention provides for methods for identifying other genes that may be sensitive to resveratrol or classes of novel compounds provided for herein comprising incubating such genes with a complementary sequence of the motif within the APO A1 promotor that is acted upon by resveratrol under hybridizing conditions and then assaying for the presence of hybridization of the complementary sequence of the motif promotor.

A further aspect of the present invention provides for methods of screening for, and identifying, synthetic compounds or neutraceuticals that may increase circulating APO A1/HDL levels in mammals. The preferred procedure for screening or identifying candidate compound(s) involves exposing permanently transfected cells Hep G2 or CaCo2 cell lines to the synthetic compounds or neutraceuticals to be screened and assaying for elevated levels of APO A1 gene transcription and/or APO A1 protein whereby such elevated transcription levels or APO A1 protein levels identify compounds or neutraceuticals capable of increasing circulating HDL levels. Other compounds, for increasing APO A1 expression could similarly be identified by incubating such compounds with permanently tansfected cell lines containing full or truncated APO A1 promotor sequences and assaying for increased APO A1 expression. The thusly identified compounds, particularly with pharmaceutically acceptable carriers would provide great clinical advantage.

A further aspect of the present invention provides for classes of novel compounds that may be used to increase transcription factor binding to egr-1 like promoter sequences, thereby modulating the expression of cancer related genes such as p21 and p53, and thereby treating cancer, and methods of treatment therewith. In addition, this approach can be extended to permit treatment of other disease conditions associated with genes controlled, at least in part, by egr-1 or egr-1 promoter like sequences as described in greater detail below.

A further aspect of the present invention to provide classes of novel compounds that may be used to increase transcription factor binding to egr-1 like promoter sequences, thereby modulating the expression of longevity related genes such as the sirtuins, and thereby extend life span of an individual so treated, and methods of treatment therewith.

A still further aspect of the present invention provides for classes of novel compounds that may be used to increase transcription factor binding to egr-1 like promoter sequences, thereby modulating the expression of cancer related genes such as p21 and p53, and thereby treating cancer, and methods of treatment therewith. In addition, this approach can be extended to permit treatment of other disease conditions associated with genes controlled, at least in part, by egr-1 or egr-1 promoter like sequences as described in greater detail below.

A further aspect of the present invention to provide classes of novel compounds of the invention that may be used to increase transcription factor binding to egr-1 like promoter sequences, thereby modulating the expression of longevity related genes such as the sirtuins, and thereby extend life span of an individual so treated, and methods of treatment therewith.

The compounds provided for in the present invention, which are presented as illustrative chemical structures, but this is not to limit the scope of the invention to the compounds listed below. When the term “nitrooxy” is used, what is meant is the nitric ester group —ONO2. When the terms “hydroxyl” or “hydroxy” are used, what is meant is the group —OH. When the term “reverse ester” is used, what is meant is the group

More particularly, the present invention provides for a compound useful for increasing transcription factor binding to egr-1 like promoter sequences comprising a stilbene compound comprising the following structure: 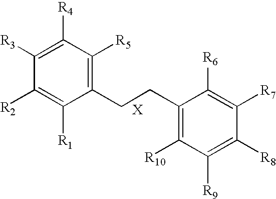
wherein
R1, R2, R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
   wherein
OCOR means 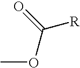
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2 and
   wherein
X can be a single, double or triple bond.

More particularly, the present invention provides for a compound useful for increasing transcription factor binding to egr-1 like promoter sequences comprising a flavonoid compound comprising the following structure: 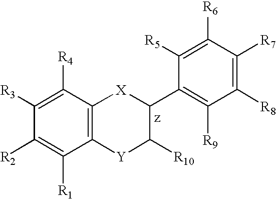
wherein
R1, R2, R3, R4, R5, R6, R7, R8, R9, R10, R13 and R14 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 or R13 or R14 is nitrooxy, R12, OR12, or OCOR12; and
   wherein
OCOR means 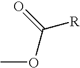
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2;
   wherein
X can be 0, CR13 or NR13;
Y can be CO [a ketone still maintaining the 6 atom ring structure], CR14 or NR14; and
Z can be a single or a double bond.

More particularly, the present invention provides for a compound useful for increasing transcription factor binding to egr-1 like promoter sequences comprising an isoflavonoid compound comprising the following structure: 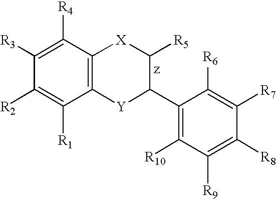
wherein
R1, R2, R3, R4, R5, R6, R7, R8, R9, R10, R13 and R14 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 or R13 or R14 is nitrooxy, R12, OR12, or OCOR12; and
   wherein
OCOR means 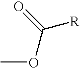
and R is R11or R12
   wherein
R11is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted optionally branched, may have one or more of the C atoms replaced by S, N or O, and optionally containing one or more ONO.sub.2;
   wherein
X can be O, CR13 or NR13;
Y can be CO [a ketone still maintaining the 6 atom ring structure], CR14 or NR14; and
Z can be a single or a double bond.

More particularly, the present invention provides for a compound useful for increasing transcription factor binding to egr-1 like promoter sequences comprising a chalcone compound comprising the following structure: 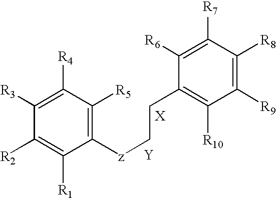
wherein
R1, R2, R3, R4, R5, R6, R7, R8, R9, R10 and R13 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 or R13 is nitrooxy, R12, OR12, or OCOR12; and
   wherein
OCOR means 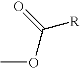
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2;
   wherein
X can be a single or a double bond;
Y can be a single or a double bond; and
Z can be CO [a ketone], CR13 or NR13;
   with the proviso that X and Y are not both double bonds, and if Z is CO then Y is not a double bond.

More particularly, the present invention provides for a compound useful for increasing transcription factor binding to egr-1 like promoter sequences comprising a polyphenol compound comprising the following structure: 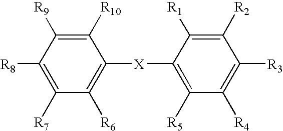
wherein
R1, R2, R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
   wherein
OCOR means 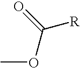
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2 and
   wherein
X can be C, S, (CO), SO, AKA ketone, (SO.sub.2)N, (CO)C, (CO)N, (CO)O, C—N [single bond], C═N [double bond], C—O, N—O, N—N [single bond], or N═N [double bond].

### BRIEF DESCRIPTION OF THE FIGURES

FIG. 1 shows a schematic map of the constructs in the transfection assays;

FIG. 2 shows the effects of resveratrol (0, 2.5, 5, 7.5 and 10 μM) on APO A1 promoter activity levels in CaCo2 cells transfected with pA1.474-Luc;

FIG. 3 shows the time course over which resveratrol (5 μM) had an effect on APO A1 levels in CaCo2 cells transfected with a reported construct, pA1.474-Luc;

FIG. 4 shows a study in CaCo2 cells transfected with different reporter constructs that contained progressively smaller fragments of the APO A1 promoter and treated with 5 μM resveratrol for 16 hours;

FIG. 5 shows a western blot analysis of APO A1 protein;

FIG. 6 shows the results of Hep G2 cells transiently tranfected with pA1.474-Luc and then treated with various doses of resveratrol for 16 hours;

FIG. 7 shows data from HepG2 cells permanently transfected with pA1.474-Luc and a commercially available neomycin resistance gene. The cells from this transfection were selected for neomycin resistance;

FIG. 8 shows the time course of the APO A1 promoter response to resveratrol in Hep G2 cells transfected with the pA1.474-Luc, exposed to 10 μM of resveratrol, and then harvested at 4, 8, 16 and 24 hrs after exposure; and

FIG. 9 shows a western blot analysis to measure the APO A1 protein content in spent media from Hep G2 cells untreated or treated with 5 or 10 μM of resveratrol.

### DETAILED DESCRIPTION OF THE INVENTION AND BEST MODE

In accordance with principles of the present invention, one aspect of the present invention provides for a method for increasing egr-1 promoters and those promoters with egr-1 consensus sequences, and thereby promote APO A1 expression; and characterizes the steps and potential mechanism in detail regarding the use of resveratrol to enhance transcription of the gene. Understanding its potential action will lead to improved development or searches for derivatives and analogues with enhanced therapeutic effect.

It is clear from the epidemiologic studies that cardiovascular disease (CVD) correlates with many parameters, but one of the most important is low levels of HDL/APO A1. Methodology that increases APO A1/HDL should reduce the risk of CVD. While hormonal regulation of APO A1 gene activity could be a way to control expression of the gene, an unfortunate accompanying disadvantage is that it is not possible to use increased concentrations of the hormones, such as thyroid hormone to up-regulate activity of the gene. Levels of thyroid hormone that exceed normal values are toxic in humans and therefore cannot be used to enhance APO A1 gene activity. Accordingly, the use of mimetics or analogues that can enhance APO A1 gene activity without the accompanying toxic effects is desired.

Compounds provided by the present invention include analogues of resveratrol, analogues of resveratrol, as well as analogues of resveratrol with attached moieties that are capable of releasing nitric oxide when administered to a patient. Such compounds include but are not limited to analogues of resveratrol wherein the nitric oxide donating moieties belong to the organic nitrate, alkoxynitrate, diazeniumdiolate, thionitroxy, and the like classes of chemical structures.

Organic nitrate (“nitroxy”) groups may be added to compounds using known nitrating agents, such as, for example, concentrated nitric acid, a mixture of nitric and sulfuric acids, or a nitric acid/acetic anhydride mixture. Alkoxynitroxy groups may be added to compounds using, for example, the methods taught in U.S. Pat. No. 5,861,246.

Diazeniumdolates may be synthesized by various methods including, or example, the methods taught in U.S. Pat. Nos. 4,954,526, 5,039,705, 5,155,137, 5,405,919 and 6,232,336, all of which are fully incorporated herein by reference.

Nitric oxide donating moieties may be advantageously attached to resveratrol or a derivative or analogue thereof via a covalent or ionic bond. Preferably, the nitric oxide donating moiety or moieties are attached by one or more covalent bonds. Nitric oxide donating moieties attached to resveratrol or an analogue or derivative thereof may be attached to any portion of the resveratrol molecule. In one embodiment, nitric oxide donating moieties are substituted in place of one or more hydroxyl groups. In a preferred embodiment, the substitutions take place on resveratrol such as natural resveratrol. In another preferred embodiment, the substitutions are of organic nitrate groups in place of hydroxyl groups. In another preferred embodiment, the nitric oxide donating moieties have replaced all three hydroxyl groups of resveratrol or a resveratrol analogue or derivative thereof.

For clarity, it is noted that the -190 to -170 region is termed “Site S”, in “Oestradiol decreases rat apolipoprotein A1 transcription via promoter site B.” Taylor et al., Journal of Molecular Endocrinology, 25(2):207-19 (2000). The −190 to −170 sequence as cited herein is considered interchangeable with Site S, The Site S sequence for rat and human APO A1 promoter regions differ by one base over this span. Rat APO A1 −190 to −170 region of the promoter is believed to comprise the nucleotide sequence “TGCAGCCCCCGCAGCTTCCTG”. The human APO A1 motif that has marked homology to the Site S is believed to comprise the nucleotide sequence “TGCAGCCCCCGCAGCTTGCTG”. The difference in the two sequences lies in a single nucleotide, which is a C in the rat and a G in the human. The human sequence is noted in Higuchi et al. 1988, JBC, 263(34):18530-6 (genbank accession M20656) and for the rat sequence Dai et al. 1990, EJB, 190(2):305-10 (genbank accession X54210). This difference in the motif is a transverse mutation.

While not wishing to be bound by any particular theory, resveratrol's activation of APO A1 expression in cells of intestinal and hepatic lineages is mediated through a consensus sequence contained within Site S. A sequence, “AGCCCCCGC”, found within Site S, ha been described as an “Egr-1 response element” consensus sequence. This motif is contained within the nucleotides spanning −196 to −174 of the human APO A1 promoter (Kilbourne et al. 1995, JBC, 270(12):7004-10). Again, without being bound by any particular theory, this AGCCCCCGC element found to be contained within Site S is a sequence through which resveratrol mediates its activity, but this is not to the exclusion of other potential required elements, resveratrol modulates APO A1 expression leading to the induction of activity in hepatocytes and intestinal cells. This is thought to be through Site S which is comprised of; in part, the AGCCCCCGC element. Resveratrol mediates activity through the AGCCCCCGC element in cells of intestinal and hepatic lineages.

It is believed that a nucleotide sequence comprising Site S or about any 8 contiguous bases of the AGCCCCCGC element act as an enhancer element when operably linked to a heterologous promoter in order to modulate the expression of a reporter gene. For example, an isolated nucleic acid comprising the −190 to −170 (or −196 to −174) region, operably linked to a promoter (for example the thymidine kinase (TK) promoter), operably linked to a reporter gene (for example luciferase, CAT, or apolipoprotein A-1 itself), in an expression system (such as CaCo2, HepG2 or other eukaryotic cells, or cellular or nuclear extracts thereof), induce measurable modulation of expression of a reporter gene when contacted with a compound whose biological activity is mediated via either Site S or the “AGCCCCCGC” element. Examples of a compound with such biological activity include resveratrol, resveratrol derivatives, resveratrol-like polyphenols, and other polyphenols (natural or synthetic). Such compounds could then act to influence egr-1 and/or egr-1 consensus sequence elements which in turn could then modulate expressions of genes associated with such enhancer elements. Consequently, this approach can then be used to effect treatment of disease or other physiological conditions associated with genes controlled, at least in part, by egr-1 or egr-1 promoter like sequences as described in greater detail below.

The steps to construct such a nucleic acid, transfect eukaryotic cells with such a nucleic acid, and assay for reporter gene expression are constructed by known protocols such as those described in Molecular cloning: a laboratory manual, by Tom Maniatis and Short Protocols in Molecular Biology, 5th Edition, Frederick M. Ausubel et al. (Editor). Such isolated nucleic acids, cells transformed with such isolated nucleic acids, methods of screening employing such cells or extracts thereof, and compounds identified by such screening methods are contemplated herein.

These isolated (recombinant) nucleic acids, the eukaryotic cells transfected with same, the screening method employing said cells or extracts thereof, and the compounds identified utilizing said screening method, are useful in the treatment of proliferative diseases, such as cancer. Examples of compounds identifiable by the screening method provided herein comprise biologically active resveratrol, resveratrol derivatives, resveratrol-like polyphenols, and other polyphenols (natural or synthetic).

Methods of Treatment Using Effectors of EGR-1 and EGR-1 Consensus Sequences

While in the following description we use the phrase “egr-1 consensus sequence elements” for convenient consistency, it is to be understood we also intend that phrase to include mediating mechanisms which work through the egr-1 site and not just those whose effect is limited to the consensus sequence. Consequently, activation or repression of egr-1 activity is to be understood to include not only action mediated through the egr-1 consensus sequence elements but also activity modulation that works directly on egr-1 or egr-1 related elements other than the consensus sequence.

Egr-1 is a key transcription factor that binds to egr-1 consensus sequence elements and which is involved in the mediation of cellular signalling from injury or stress induced events to effector genes, some of which assist in the repair or apoptosis of the injured tissue, and other of which are linked to the pathophysiology and pathogenesis of disorders arising from the inductive lesion. Stressors or injuries that may alter the activation of events that are mediated through egr-1 consensus sequence elements include shear stress, ultraviolet light induced damage, hypoxia, radical oxygen species, angiotensin II, platelet derived growth factors, acidic fibroblast growth factor (FGF-1) and additional mechanical and non-mechanical injuries and stresses.

Once activated, egr-1 alters, either by increasing or decreasing, the transcription levels of numerous downstream genes including PDGF-A, PDGF-B, FGF-2, apolipoprotein A1, macrophage colony-stimulating factor (M-CSF), TNF-α, tissue factor, urokinase-type plasminogen activator (u-PA), interleukin-2 (IL-2), intercellular adhesion molecule-1 (ICAM-1), copper-zinc superoxide dismutase gene (SOD I), p53, thrombospondin, CD44, and 5-lipoxygenase (5-LO), and peroxisome proliferator-activated receptor-1 (PPAR-1). Obviously, many of these genes are compelling therapeutic targets, such as M-CSF for leukocyte proliferation associated disorders, apolipoprotein A1, PPAR and 5-LO for cholesterol associated disorders, ICAM-1 for cellular adhesion associated disorders including cancer, SOD 1 for hyper or hypo-oxidation associated disorders and others that will be readily apparent to those of skill in the at.

Egr-1 involvement in trans-activation of target genes is affected by the number, location, and degree of homology of egr-1 consensus sequence sites in the promoter region of the target gene, by the adjacent DNA binding motifs of other trans-activating factors, by direct interactions with other activators and/or repressors, the cell type in which the egr-1 activation occurs, and by the state of phosphorylation of egr-1. Modulation of egr-1 expression, therefore, can lead to either activation or repression of a target gene.

Compounds Capable of Effecting Modulation of EGR-1 Expression

Compounds provided by the present invention include analogues of resveratrol, other stilbenes, other polyphenols, and flavonoids, with attached moieties that are capable of releasing nitric oxide when administered to a patient. Such compounds include but are not limited to analogues of resveratrol, other stilbenes, other polyphenols, and flavonoids, wherein the nitric oxide donating moieties belong to the organic nitrate, alkoxynitrate, diazeniumdiolate, thionitroxy, and the like classes of chemical structures.

An understanding of the exact mechanisms by which alteration of the compounds of the invention is not required to practice the present invention. The mechanism disclosed herein are intended to be non-limiting and serve only to better describe the present invention. While not being limited to a theory, resveratrol is believed to cause the previously described effects due to its molecular structure, the reactive and necessary core consisting of at least one aromatic ring structure, with at least one hydroxyl group located on an aromatic ring. Naturally produced resveratrol itself is specifically comprised of two aromatic rings, with two hydoxyls located at the 3 and 5 positions on one ring and one hydroxyl located at the 4′ position on the other, and the two aromatic rings are connected by two carbon atoms which have a double bond between them. Other compounds of this general class, said class being those compounds which comprise at least one aromatic ring structure with at least one hydroxyl group located on the ring, are believed to possess the same capabilities and to produce the same results as those listed for resveratrol.

Consequently, stilbenes, which comprise two aromatic rings linked by two carbon atoms, other polyphenols, such as those comprising two or more aromatic rings, preferably two, linked by one, two or tree atoms, said atoms independently selected from the group consisting of nitrogen carbon, oxygen and sulfur, and which may or may not be independently substituted with side groups such as ketone oxygens, and flavonoids, such as but not limited to naturally occurring flavonoids, such as but not limited to naringenin, quercetin, piceatannol, butein, fisetin, isoliquiritgenin, and hesperitin, are all compounds possess similar properties as those described for resveratrol. As a result, it has been discovered that any of these compounds may be considered to be functionally interchageable with resveratrol when utilized for the prevention or treatment of diseases, disorder or conditions, especially but not limited to those diseases, disorders or conditions associated with cholesterol, cardiovascular disease, hypertension, oxidative damage, dyslipidemia, apolipoprotein A1 or apoB regulation, or in modifying or regulating other facets of cholesterol metabolism such as inhibiting HMG CoA reductase, increasing PPAR activity, inhibiting ACAT, increasing ABCA-1 activity, increasing HDL, or decreasing LDL or triglycerides. Flavonoids that do not have nitric oxide donating moieties attached have previously been taught as having potential serum cholesterol reducing activities, for example in U.S. Pat. Nos. 5,877,208, 6,455,577, 5,763,414, 5,792,461, 6,165,984, and 6,133,241.

Similarly, any of the stilbenes, polyphenols, isoflavanoids, chalcones and flavonoids of this class may be considered to be functionally interchangeable with resveratrol when utilized to modulate transcription from site S, from the AGCCCCCGC element, or when utilized to inhibit leukocyte adhesion or platelet aggregation, or to inhibit COX-1, This is not to imply that all of the compounds win be identical in terms of the level of activity for each of these functions or capabilities, or for in vivo toxicity or efficacy, or for bioavailability. These compounds demonstrate, over tho course of simple testing, easily performed by one of skill in the art and not requiring undue experimentation, that some provide improved capabilities or functionality relative to others, and are therefore preferred over others as therapeutic agents.

As well, it is known that phenolic hydroxyl groups, such as those found in the base compounds upon which the present invention improves, are prone to glucoronidation and sulfation reactions that facilitate excretion. Protection against these reactions by blocking the phenolic hydroxyl group with another chemical group, such as a nitric ester (also referred to as an organic nitrate or ONO.sub.2) group, alkoxy nitroxy, or reverse ester nitrooxy (nitrooxy groups are also referred to as nitro oxy groups) further extends a molecule's half life in the body and postpones excretion.

As an example, resveratrol, which contains three putatively important and therapeutically active hydroxyl groups, may be protected by the replacement of the hydroxyl groups with nitric esters (also known as nitrates, nitroxy groups, or ONO.sub.2 and are occasionally referred to as nitroxy, but which should not be confused with NO.sub.2) alkoxy nitrooxy groups, or reverse ester nitrooxy groups which are replaced over time while in the body with hydroxyl groups to reconstitute the active compound, resveratrol. As the nitric oxide donating groups are replaced with hydroxyl groups one at a time over a period, and the resveratrol molecule comprising one or two nitric oxide donating groups is still partially active, the effective half life in the body of resveratrol activity is increased. Such a strategy further permits the use of lower doses of the nitrate form of resveratrol relative to the parent, hydroxylated form of resveratrol, which then results in lower side effects in the patient. Obviously, such an approach would also be effective for the other stilbenes, polyphenols, isoflavanoids, chalcones and flavonoids contemplated in the invention as they also are contemplated to comprise one or more hydroxyl groups that may form an integral part of the molecule's active site.

The present invention provides for the synthesis, composition and methods of treatment for nitrooxy derivatives of compounds other than the above described stilbenes, polyphenols, isoflavanoids, chalcones and flavonoids; wherein said compounds, which may be a nitrooxy derivative are synthesized and contain aromatic or heteroaromatic ring, one or more hydroxyl groups, and are known to modulate serum cholesterol levels. One example class of compounds that contain aromatic or heteroaromatic rings, one or more hydroxyl groups, and are known to modulate serum cholesterol levels comprise HMG CoA reductase inhibitors, also known as statins. Commercially available statins, the nitrooxy derivatives of which are provided for in this invention, comprise atorvastatin, lovastatin, pravastatin, simvastatin, fluvastatin, cerivastatin, and rosuvastatin. Two other compounds that fall within the specification of containing aromatic or heteroaromatic rings, one or more hydroxyl groups, and known to modulate serum cholesterol levels are ezetimibe and niacin. The nitrooxy derivatives of ezetimibe and niacin are therefore also provided for in this invention.

Synthesis of Nitric Oxide Donating Derivatives of Stilbenes, Polyphenols, Flavonoids, Statins and Ezetimibe

Organic nitrate (also referred to as nitrooxy, nitric esters, ONO.sub.2 and occasionally as “nitroxy” but which is not to be confused with NO.sub.2) groups may be added to compounds using known methods, such as that of Hakimelahi wherein the nitrooxy group is substituted for existing hydroxyl groups on the parent molecule (Hakimelahi et al. 1984. Helv. Chim. Acta. 67:906-915).

Alkoxynitroxy groups may be added to compounds using, for example, the methods taught in U.S. Pat. No. 5,861,426. Diazeniumdolates may be synthesized by various methods including, for example, the methods taught in U.S. Pat. Nos. 4,954,526, 5,039,705, 5,155,137, 5,405,919 and 6,232,336, all of which are fully incorporated herein by reference.

Nitric oxide donating moieties may be advantageously attached to a stilbene, such as resveratrol, a polyphenol, or a flavonoid, such as naringenin, or other compounds as described and provided for in this invention, such as a member of the class of statins, or a derivative or analogue thereof via a covalent or ionic bond. Preferably, the nitric oxide donating moiety or moieties are attached by one or more covalent bonds. Nitric oxide donating moieties may be advantageously attached to any portion of the molecule. In one embodiment, nitric oxide donating moieties are substituted in place of one or more hydroxyl groups. In a preferred embodiment, the substitutions are of organic nitrate groups in place of hydroxyl groups. In another preferred embodiment, the substitutions are of organic nitrate groups attached to esters or to reverse esters in place of hydroxyl groups. In another preferred embodiment, the nitric oxide donating moieties have replaced all of the hydroxyl groups of the stilbene, such as resveratrol, the polyphenol or the flavonoid, such as naringenin, or other compounds as described and provided for in this invention, such as any member of the class of stains, or those hydroxyl groups of an analogue or darivative thereof.

For all of the compounds of the invention, substitution of a hydroxyl group by a fluoride ion, a chloride ion, a bromide ion, a CF.sub.3 group, a CCl.sub.3 group, a CBr.sub.3, an alkyl chain of 1 to 18 carbon atoms, optionally substituted, optionally branched, or an alkoxy chain of 1 to 18 carbon atoms, optionally substituted, optionally branched is also contemplated and provided for, as such modifications to patent compounds are commonplace, known to increase drug stability without altering the mechanism of action, and are readily accomplished by one of skill in the art.

For all of the compounds of the invention, acetylated-derivatives of the compounds are also contemplated and provided for, as such modifications to parent compounds are commonplace, known to improve the beneficial effects of the drag without altering the mechanism of action, and are readily accomplished by one of skill in the art. Acetylated derivatives include esters, reverse esters, esters with nitric oxide donating moieties (including but not limited to nitrooxy groups) attached, and reverse esters with nitric oxide donating moieties (including but not limited to nitrooxy groups) attached.

For all of the compounds of the invention, phosphorylated-derivatives of the compounds are also contemplated and provided for, as such modifications to parent compounds are commonplace, known to improve the beneficial effects of the drug without altering the mechanism of action, and are readily accomplished by one of skill in the art.

Glucoronidated derivatives of the compounds contemplated by the invention are also contemplated herein, as glucoronidation is a process that naturally occurs in the body as part of the metabolism of stilbenes, other polyphenols, and flavonoids. Once provided to a patient, many of the compounds of the invention will be modified in the body and will therefore be present in the body in glucoronidated form. The conjugation of glucoronic acid to the compounds of the invention prior to administration will therefore not preclude the function or therapeutic utility of the compounds as determined by in vivo studies. As a result, compounds of the invention with an additional sugar moiety attached are considered to be functionally comparable to the parent compounds, and are therefore provided for in the present invention. Glucoronidation of any stilbene, polyphenol or flavonoid derivative compound contemplated by the present invention may be achieved, for example, using human liver microsomes as in the method of Otake (Otake et al. Drug Metab Disp 30:576 (2002)).

Similarly, sulfated derivatives of the compounds contemplated by the invention are also contemplated herein, as sulfation is a process that naturally occurs in the body as part of the metabolism of stilbenes, other polyphenols, and flavonoids. Once provided to a patient, some of the compounds of the invention will be modified in the body and will therefore be present in the body in sulfated form. Sulfation will therefore not preclude the function or therapeutic utility of the compounds as determined by in vivo studies. As a result, compounds of the invention that have been subjected to a sulfation reaction are considered to be functionally comparable to the parent compounds, and are therefore provided for in the present invention. Sulfation of any stilbene, polyphenol or flavonoid derivative compound contemplated by the present invention may be achieved, for example, using the ion-air extraction method of Varin (Varin et al Anal Biochem 161:176 (1987)).

Salts of the compounds described herein, including those preferred for pharmaceutical formulations, are also provided for in this invention.

Compounds Contemplated by the Invention

In order to clarify, the compounds provided for in the present invention are presented as illustrative chemical structures, but this is not to limit the scope of the invention to the compounds listed below. When the term “nitrooxy” is used, what is meant is the nitric ester group —ONO₂. When the terms “hydrooxy” or “hydroxy” are used, what is meant is the group —OH. When the term “reverse ester” is used, what is meant is the group 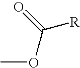
wherein die O-bond is to the parent compound of flavonoid, stilbene or polyphenolic structure and R is C₁₋₁₈, aryl, heteroaryl or a derivative thereof, wherein said derivative is optionally substituted, optionally branched, and may have one or more of the C atoms replaced by S, N or O.

When the term “reverse ester nitro oxy” is used, what is meant is the group 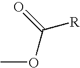
wherein the O-bond is to the parent compound of flavonoid, stilbene or polyphenolic structure and R is C₁₋₁₈, aryl, heteroaryl or a derivative thereof, wherein said derivative is optionally substituted, optionally branched, and may have one or more of the C atoms replaced by S, N or O, and containing one or more ONO.sub.2.

The present invention provides for compounds useful for increasing transcription factor binding to egr-1 like promoter sequences having the general stilbene stricture: 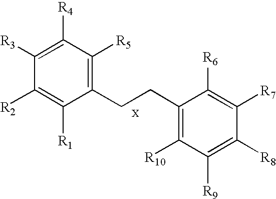
which can be further subdivided into the following structures: 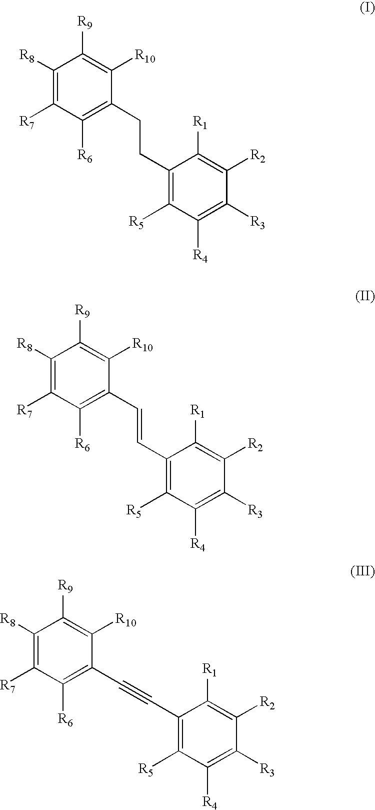
   wherein
R1, R2, R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
   wherein
OCOR means 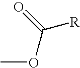
and R is R11or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2

The present invention also provides for compounds useful for increasing transcription factor binding to egr-1 like promoter sequences of the following general structures: 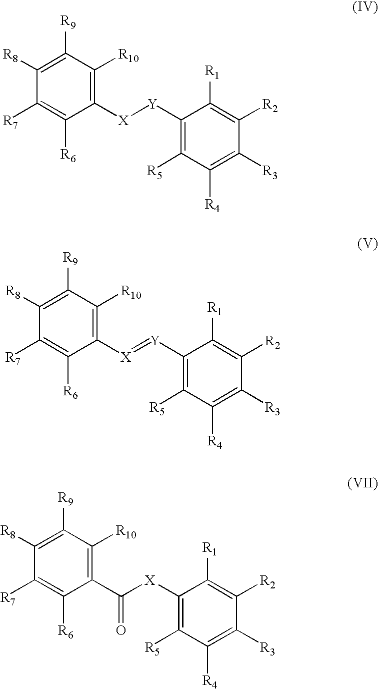
wherein
R1, R2, R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
   wherein
OCOR means 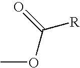
and R is R11or R12
   wherein
R11is C₁₋₁₈, aryl heteroaryl or a derivative thereof, wherein said derivative is optionally substituted and optionally branched, and may have one or more of the C atoms replaced by S, N or O, and
   wherein
R12 in C₁₋₁₈, aryl, heteroaryl or a derivative thereof, wherein said derivative is optionally substituted, optionally branched, may have one or more of the C atoms replaced by S, N or O, and optionally containing one or more ONO.sub.2
   Wherein
X and Y may each independently be C, N, O, with the proviso that if either of X or Y is C then the other is not C.

The present invention also provides for compounds useful for increasing transcription factor binding to egr-1 like promoter sequences of the following general structure: 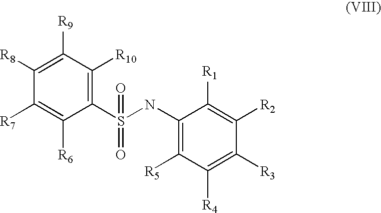
wherein
R1, R2, R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitroxy, R12, OR12, or OCOR12; and
   wherein
OCOR means 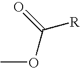
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2

The present invention also provides for compounds useful for increasing transcription factor binding to egr-1 like promoter sequences having the general polyphenol structure: 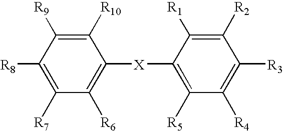
which can be further subdivided into the following structures: 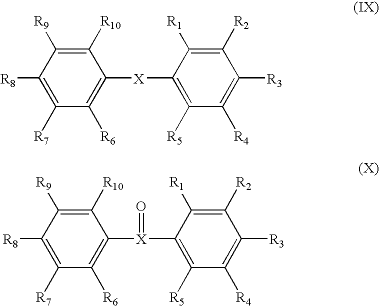
Wherein
X is C or S
   Wherein
R1, R R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
   wherein
OCOR means 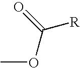
and R is R11 or R12
   wherein
R11is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2

The present invention also provides for compounds useful for increasing transcription factor binding to egr-1 like promoter sequences having the general flavonoid structure: 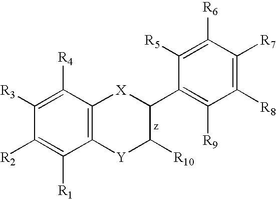
which can be further subdivided into the following structures:

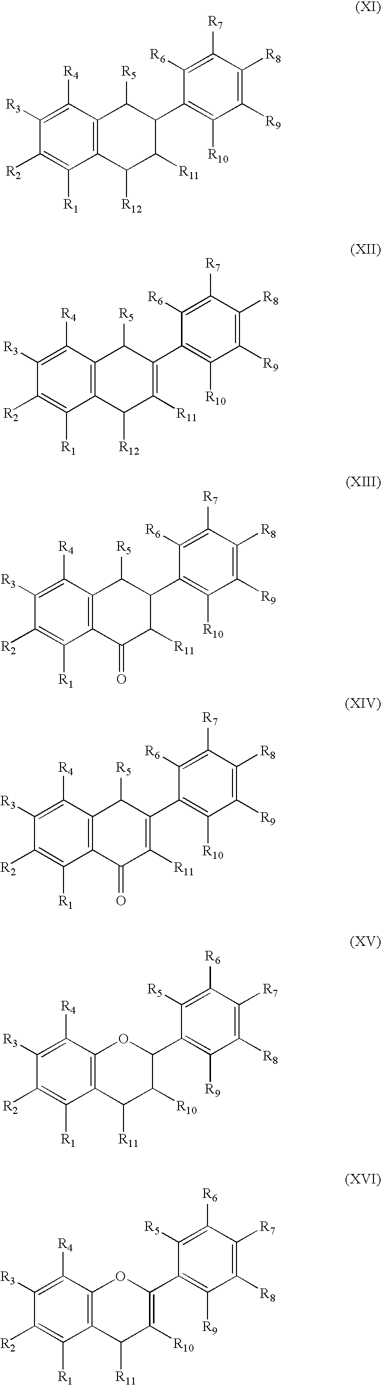

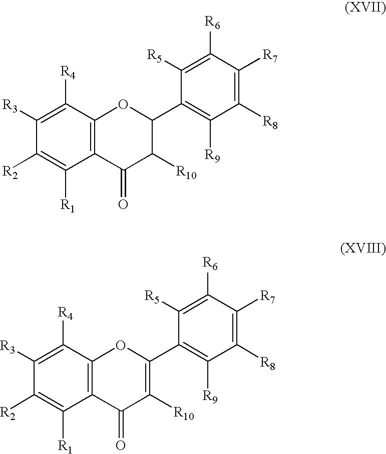

The present invention also provides for compounds useful for increasing transcription factor binding to egr-1 like promoter sequences having the general isoflavonoid structure: 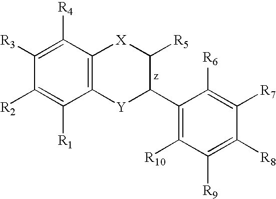
which can be further subdivided into the following structures: 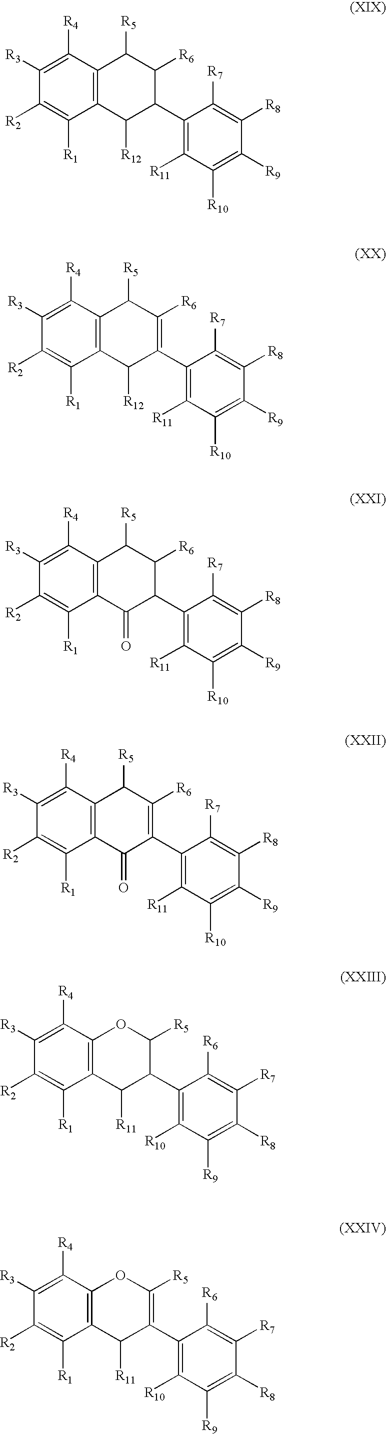 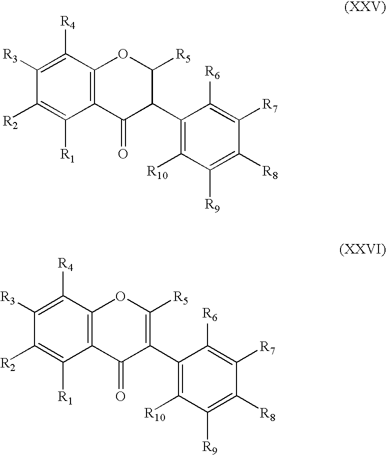
wherein
R1, R2, R3, R4, R5, R6, R7, R8, R9, R10, R11, R12, R15, and R16 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R13, R14, OR13, OR14, OCOR13, OCOR14, O-sulfate [the sulfate conjugate], or O-glucoronidate [the glucoronic (AKA glucuronic) acid conjugates], with the proviso that at least one of R1-R12 or R15 O R16 is nitrooxy, R14, OR14, or OCOR14; and
   wherein
OCOR means 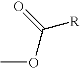
and R is R13 or R14
   wherein
R13 is C₁₋₁₈, aryl, heteroaryl or a derivative thereof, wherein said derivative is optionally substituted and optionally branched, and may have one or more of the C atoms replaced by S, N or O, and
   wherein
R14 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2;
   wherein
X can be O, CR15 or NR15;
Y can be CO [a ketone still maintaining the 6 atom ring structure], CR16 or NR16; and
Z can be a single or a double bond,

The present invention also provides for compounds useful for increasing transcription factor binding to egr-1 like promoter sequences having the general chalcone structure: 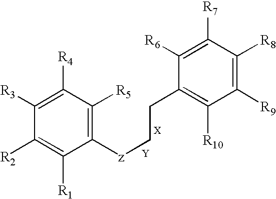
some structures of which are represented by the following structures 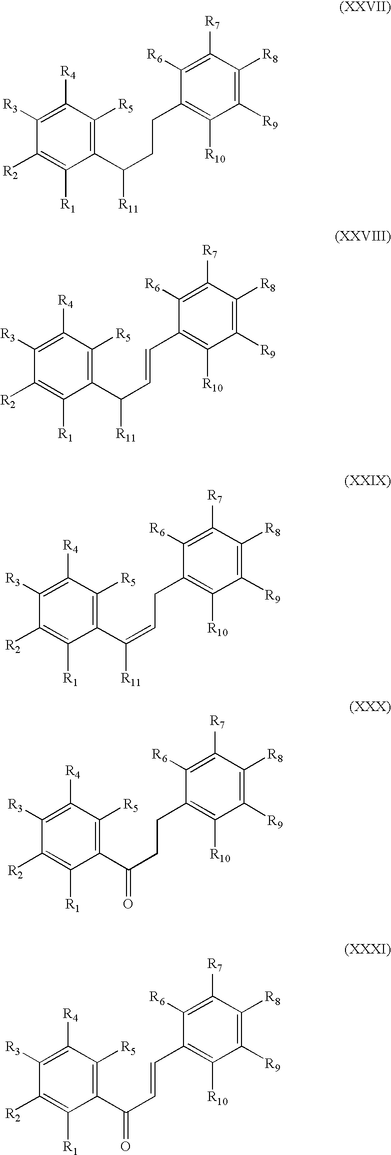
wherein
R1, R2, R3, R4, R5, R6, R7, R8, R9, R10, and R11 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R13, R12, OR13, OR12, OCOR13, OCOR12, O-sulfate [the sulfate conjugate], or O-glucoronidate [the glucoronic (AKA glucuronic) acid conjugates], with the proviso that at least one of R1-R11 is nitrooxy, R12, OR12, or OCOR12; and
   wherein
OCOR means 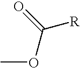
and R is R12 or R13
   wherein
R13 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2; and
   wherein
X can be a single or a double bond;
Y can be a single or a double bond; and
Z can be CO [a ketone], CR11 or NR11.

The present invention also provides for compounds useful for increasing transcription factor binding to egr-1 like promoter sequences of the following general formula: 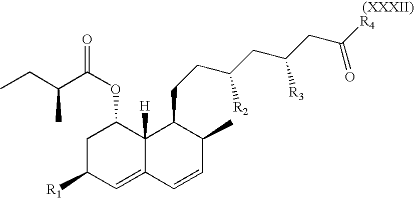
wherein
R1, R2, R3, R4 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R4 is nitrooxy, R12, OR12, or OCOR12; and
   Wherein
OCOR means 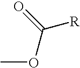
and R is R11 or R12
   wherein
R11 is C₁₋₁₈, aryl heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for the compound useful for increasing transcription factor binding to egr-1 like promoter sequences comprising: 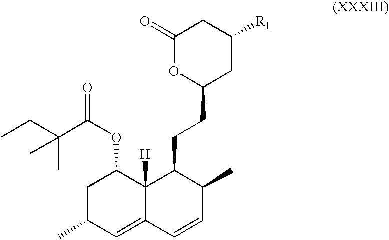
wherein
R1 is nitrooxy, R12, OR12, or OCOR12; and
   wherein
OCOR means 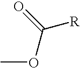
and R is R12
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for the compound 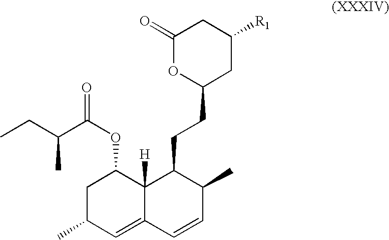
wherein
R1 is nitrooxy, R12, OR12, or OCOR12; and
   wherein
OCOR means 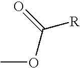
and R is R12
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for compounds useful for increasing transcription factor binding to egr-1 like promoter sequences of the following general formulae 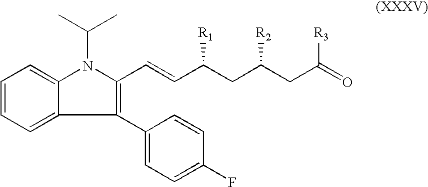
wherein
R1, R2, R3 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R3 is nitrooxy, R12, OR12, or OCOR12; and
   wherein
OCOR means 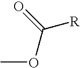
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for compounds of the following general formulae 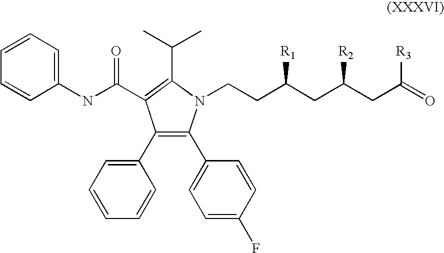
wherein
R1, R2, R3 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R3 is nitrooxy, R12, OR12, or OCOR12; and
   Wherein
OCOR means 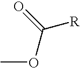
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for compounds useful for increasing transcription factor binding to egr-1 like promoter sequences of the following general formulae 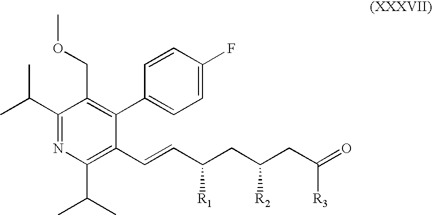
wherein
R1, R2, R3 may each be independently hydrogen, hydroxyl [OH], hydroxyalkyl, aminoalkyl Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucoronic) acid conjugates], with the proviso that at least one of R1-R3 is nitrooxy, R12, OR12, or OCOR12; and
   Wherein
OCOR means 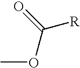
and R is R11 or R12
   wherein
R11 is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for compounds useful for increasing transcription factor binding to egr-1 like promoter sequences of the following general formulae 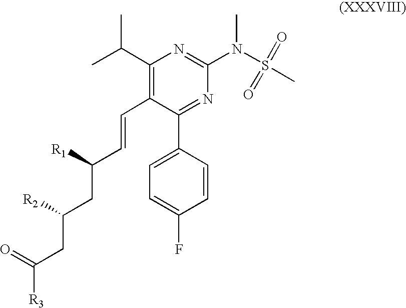
wherein
R1, R2, R3 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R3 is nitrooxy, R12, OR12, or OCOR12; and
   wherein
OCOR means 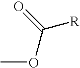
and R is R11 or R12
   wherein
R11is C₁₋₁₈, aryl, heteroaryl or a derivative thereof, wherein said derivative is optionally substituted and optionally branched, and may have one or more of the C atoms replaced by S, N or O, and
   wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2.

The present invention also provides for compounds useful for increasing transcription factor binding to egr-1 like promoter sequences of the following general formula 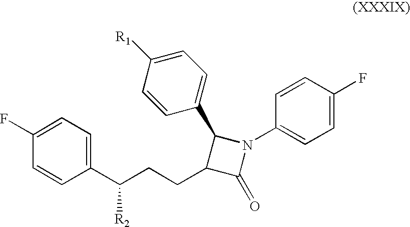
wherein
R1, R2 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R2 is nitrooxy, R12, OR12, or OCOR12; and
   wherein
OCOR means 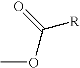
and R is R11 or R12
   wherein
R11 is C₁₋₁₈, aryl, heteroaryl or a derivative thereof, wherein said deivative is optionally substituted and optionally branched, and may have one or more of the C atoms replaced by S, N or O, and
   wherein

R12 is C₁₋₁₈s, aryl, heteroaryl or a derivative thereof, wherein said derivative is optionally substituted, optionally branched, may have one or more of the C atoms replaced by S, N or O, and optionally containing one or more ONO.sub.2.

The present invention also provides for the compound useful for increasing transcription factor binding to egr-1 like promoter sequences comprising: 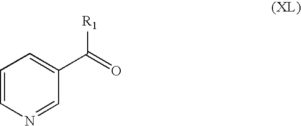
wherein
R1 is nitrooxy, R12, OR12, or OCOR12; and
   wherein
OCOR means 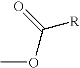
and R is R12
   wherein
R12 is C₁₋₁₈, aryl, heteroaryl or a derivative thereof, wherein said derivative is optionally substituted, optionally branched may have one or more of the C atoms replaced by S, N or O, and optionally containing one or more ONO.sub.2.
   Methods for the Synthesis of No-donating Derivatives of Stilbenes, Polyphenols and Flavonoids

It will be readily apparent to one skilled in the art that numerous methods exist for the synthesis of nitric oxide donating analogues or derivatives of stilbenes, such as resveratrol, polyphenols, or flavonoids, such as naringenin, or of other anti-oxidant, serum cholesterol decreasing or reverse cholesterol transport activating or HDL increasing compounds. Despite the existence of known methods, no such compounds have ever been described or synthesized before. Preferably, such compounds would be analogues or derivatives of stilbenes, such as resveratrol, of polyphenols, or of flavonoids, such as naringenin, or of other anti-oxidant, serum cholesterol decreasing or reverse cholesterol transport activating or HDL increasing compounds bound to nitric oxide donating moieties. Most preferably, such compounds would be analogues or derivatives of stilbenes, such as resveratrol, polyphenols, or flavonoids, such as naringenin, or of other anti-oxidant, serum cholesterol decreasing or reverse cholesterol transport activating or of HDL increasing compounds with one or more ONO.sub.2 groups, also referred to as nitric esters, organic nitrates, or nitrooxy groups, replacing hydroxyl groups of the parent compound.

An example of a compound provided for by the present invention is resveratrol substituted with organic nitrate groups in place of the three hydroxyl groups present on naturally occurring resveratrol. This compound would be named 3, 4′, 5 trinitrooxy trans stilbene, or resveratrol trinitrate, or using IUPAC nomenclature, 1,3-BIS-nitrooxy-5-[2-(4-nitrooxy-phenyl)-vinyl)-benzene. Another example of such a compound provided for by the present invention is naringenin substituted with organic nitrate groups in place of the tree hydroxyl groups present on naturally occurring naringenin. This compound would be named naringenin trinitrate, or using IUPAC nomenclature, 5,7-bis-nitrooxy-2-(4-nitrooxy-phenyl)-chroman-4-one. Another example of a compound provided for by the present invention is the reverse ester nitrooxy analogue of Naringenin, which with three hydroxyls substituted would be 5-Nitrooxy-pentanoic acid 4-[5,7-bis-(5-nitrooxy-pentanoyloxy)-4-oxo-chroman-2-yl]-phenyl ester. While not being limited to those compounds explicitly described herein, many more examples are provided in the example section of the present invention.

The trans-resveratrol source material to be used in the reaction could be obtained commercially from Bio-Stat Limited (Stockport, U.K.) or Sigma Chemical Co. (St. Louis, Mo., USA), isolated from wine using the procedure of Goldberg et al. (1995) Am. J. Enol. Vitic. 46(2):159-165. Alternatively, trans-resveratrol may be synthesized according to the method of Toppo as taught in U.S. Pat. No. 6,048,903 or from appropriately substituted phenols by means of a Wittig reaction modified by Waterhouse from the method of Moreno-Manas and Pleixats.

The naringenin to be used as an ingredient for synthesis reactions is a naturally occurring compound readily available from numerous commercial sources, or alternatively, isolatable using well known methods requiring no undue experimentation from natural sources such as citrus juice.

Administration

For treatment of the conditions referred to above the compounds may be used per se, but more preferably are presented with an acceptable carrier or excipient in the form of a pharmaceutically acceptable formulation. These formulations include those suitable for oral, rectal, topical, buccal and parenteral (e.g. subcutaneous, intramuscular, intradermal, or intravenous) administration, although the most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of the compound as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association the active compound and the carrier or excipient (which may constitute one or more accessory ingredients). The carrier must be acceptable in the sense of being compatible with the other ingredients of the formulation and must not be deleterious to the recipient. The carrier may be a solid or a liquid, or both, and is preferably formulated with the compound as a unit-dose formulation, for example, a tablet, which may contain from 0.05% to 95% by weight of the active compound. Other pharmacologically active substances may also be present including other compounds. The formulations of the invention may be prepared by any of the well known techniques of pharmacy consisting essentially of admixing the components.

For solid compositions, conventional nontoxic solid carriers include, for example, pharmaceutical grades of mannitol, lactose, starch, magnesium stearate, sodium saccharin, tale, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. In general, suitable formulations may be advantageously prepared by uniformly and intimately admixing the active compound with a liquid or finely divided solid carrier, or both, and then, if necessary, shaping the product. For example, a tablet may be prepared by compressing or molding a powder or granules of the compound, optionally with one or more assessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Molded tablets may be made by molding, in a suitable machine, the powdered compound moistened with an inert liquid diluent.

Formulations suitable for buccal (sub-lingual) administration include lozenges comprising a compound in a flavored base, usually sucrose and atacia or tragacanth, and pastilles comprising the compound in an inert base such as gelatin and glycerin or sucrose and acacia.

Formulations of the present invention suitable for parenteral administration comprise sterile aqueous preparations of the compounds, which are approximately isotonic with the blood of the intended recipient. These preparations are administered intravenously, although administration may also be effected by means of subcutaneous, intramuscular, or intradermal injection. Such preparations may conveniently be prepared by admixing the compound with water and rendering the resulting solution sterile and isotonic with the blood. Injectable compositions according to the invention will generally contain from 0.1 to 5% w/w of the active compound.

Formulations suitable for rectal administration are presented as unit-dose suppositories. These may be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers and excipients which may be used include Vaseline, lanoline, polyethylene glycols, alcohols, and combinations of two or more thereof. The active compound is generally present at a concentration of from 0.1 to 15% w/w of the composition, for example, from 0.5 to 2%.

The amount of active compound administered will, of course, be dependent on the subject being treated, the subject's weight, the manner of administration and the judgment of the prescribing physician. In the method of the invention a dosing schedule will generally involve the daily or semi-daily administration of the encapsulated compound at a perceived dosage of 1 ug to 1000 mg. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

### EXAMPLES

The following examples are set forth to assist in understanding the invention and should not be construed as specifically limiting the invention described and claimed herein. Such variations of the inventions which would be within the purview of those skilled in the art, including the substitution of equivalent compounds now known or later developed, including changes in formulation or minor changes in experimental design, are to be considered to fall within the scope of the invention incorporated herein.

For all the examples provided herein, unless otherwise noted the term “the compounds” or “the compound” will refer to any of the compounds provided for in the present invention. Without limiting the scope of the examples, representative compounds include 3, 4′, 5 trinitroxy trans stilbene, 3, 4′, 5 tri(nitroxy)ethoxy trans stilbene and the diazeniumdiolate derivative of trans resveratrol wherein one or both of the carbon atoms that link the two phenyl rings are substituted with nitrogen atoms that have diazeniumdiolate groups attached.

All examples listed herein were performed using the following processes and methodologies, and refer to the following, except where otherwise stated.

Cell Culture

Human hepatoblastoma cells (HepG2) and intestinal cells (CaCo2) were obtained from the American Type Culture Collection (Rockville, Md.). Cells were grown in Minimum Essential Medium (MEM) (Gibco) supplemented with 2 mM glutamine, MEM vitamin solution and 10% fetal bovine serum (FBS) for HepG2 and 20% FBS (Gibco) for CaCo2 cells. All cells were incubated in a 95% air/5% CO₂ atmosphere.

Plasmids

The plasmids created for the studies contained the human APO A1 promoter from −474, −375, −325, −235, −190 to −170 fused to the firefly luciferase gene in the vector, pGL3 (Promega). Insertion of the promoter DNA was verified by nucleotide sequence analysis. Plasmid DNA was prepared from bacteria containing the desired clone and isolated using Qiagen kits according to manufacturer's instructions and used in the tansfection studies or to create a stable cell line.

Cell Treatments

The CaCo2 or HepG2 cells were grown in the defined media and, for promoter assay studies, transfected with the reporter construct of interest. Cells were then left in serum-free media for 8-12 hours after which time resveratrol was added to media to give a final concentration of the agent as stated in the figure legends. The cells were exposed to the agent for varying periods of time, harvested and then the parameter of interest, either APO A1 protein or promoter activity, was assayed.

Transient/Permanent Transfections

For transient transfections cells were seeded onto six well plates and grown to 30-40% confluence, The cells were then transfected using 5 μl of Superfect (Qiagen) and up to one microgram of the plasmid of interest in 100 μl of serum and antibiotic free MEM. The solution was incubated for 10 minutes at room temperature. Media was then removed from the cells to be transfected and 1 ml of media was added to the DNA-Superfect mixture before being applied to the cells. The cells were then exposed to the DNA for 2 hours at 37° C./5% CO₂ and then the media containing DNA was removed and replaced with serum free MEM media allowed to grow over night prior to harvest.

HepG2 cells were also permanently transfected with 474-luciferase using a co-transfection method. Hep G2 cells are grown in MEM (Gibco) and 10% fetal calf serum (Gibco) and then co-transfected with 474-Luc along with another plasmid that carries neomycin resistance. Then 400-600 μg per ml of neomycin was added to the media and the cells surviving treatment with neomycin assayed for Luc-activity, which when present demonstrates the cells have been permanently transfected.

Preparation of Cell Lysate for Luciferase and Beta-Galactosidase Assays.

Cells were transfected with CAT plasmid of interest (see above) along with 0.5 μg of Rous sarcomavirus-βgalactosidase (RSV-beta-Gal) to monitor the efficiency of DNA uptake by cells. All cells were then left in serums poor media for 12 hours before treatment with resveratrol (Calbiochem) for various periods of time. Harvested cells were then lysed using a commercially available reporter lysis buffer (Promega) and cellular debris was collected at 13,000 rpm for 5-minutes. Aliquots of the supernatant were taken for measurement of β-galactosidase activity (Promega) and for total protein determination using Bradford Assay (Bio-Rad reagent).

Measurement of Luciferase Activity

Cells were transfected with Luciferase plasmid of interest (see above) and left to recover overnight in serum poor media. These cells or those that were permanently transfected with the luciferase promoter were then treated with varying concentrations of resveratrol for stated periods of time. As above, RSV-beta-Gal was co-transfected as a control to normalize for DNA uptake. Cells were then harvested and suspended in reporter lysis buffer (Promega). A 10 μl aliquot of this lysate was used for determination of luciferase activity, and 5 μl were used for total protein determination (Bradford Assay, Bio-Rad reagent). Luciferase activity was then determined and expressed relative to the protein concentration of that sample.

Western Blotting

Media or cells were harvested from untreated and treated HepG2/CaCo2 culture dishes at various time points and stored at −80° C. when required. For experiments in which media was collected for western blotting, cells from these dishes were trypsinized (Gibco) and a 100 μl sample of cells was used to determine the percentage of dead cells by counting live/dead cell ratios using coomasie blue staining. The remaining cells were then assessed for total DNA content using method described by Maniatis, (Cloning Manual). DNA content per dish was then utilized along with ratio of live/dead cells to normalize the amount of media to be separated by polyacrylamide gel electrophoresis. For experiments requiring western blot of whole cell lysates, cells were harvested and lysed using reporter lysis reagent (Promega) and cell debris was spun down at 13,000 rpm for 5 minutes. An aliquot of the supernatant was then used to determine amount of protein per samples were using Bradford assay (Rio-Rad reagent). Equal amounts of protein from all samples were then separated by polyacrylamide gel electrophoresis as was done with media. The gels were then transferred to nitrocellulose membrane (Hybond, Amersham Pharmacia Biotech), which was then probed with a monoclonal antibody against human APO A1 (Calbiochem).

Immunofluorescence Labeling of APO A1

HepG2 and CaCo2 cells were grown on cover slips. Cover slips on which CaCo2 cells were grown were also coated with fibronectin (Calbiochem). After treatments with various amounts of ethanol or resveratrol for 24 or 48 hours, the cells were fixed and permeabilized with a solution containing a mixture of 3.7% formaldehyde, 0.25% glutaraldehyde and 0.25% triton-X in PEM buffer (160 mmol/L PIPES, 10 mmol/L egtazic acid (EGTA), 4 mmol/L MgC12, pH 6.9) for ten minutes at room temperature. After washing three times with phosphate-buffered saline (PBS) the cells were treated with the reducing agent sodium borohydride, 1 mg/ml in PBS for 3×5 minutes. The cells where then washed again in PBS. Mouse monoclonal anti-APO A1 antibody (Calbiochem) was diluted 1:50 with PBS and added to each coverslip and incubated in a humid chamber for 60 minutes at room temperature. After washing, the FITC-conjugated secondary antibody (goat anti-mouse IgG, Jackson ImmunoResearch) was diluted 1:200 with PBS and added to coverslips for 45-60 minutes at room temperature. Cells were then given a final wash with PBS and mounted on glass slides using mounting media containing P-phenylene diamine and 50% glycerol in PBS. The FITC-labeled ApoA1 peptide in cells was visualized using a Zeiss fluorescence microscope (Zeiss, Dusseldorf, Germany) with FITC excitation and emission wavelengths of 488 and 520 nm. Photographs were taken using a Kodak digital camera mounted onto the microscope. Exposure times were identical for both treated and untreated cells. Final magnification was 250×.

### EXAMPLE 1

### Preparation of 1,3-BIS-nitrooxy-5-[2-(4-nitrooxy-phenyl)-vinyl)-benzene.

To a solution of 1 mmol of 5-[(E)-2-(4-hydroxy-phenyl)-vinyl]-benzene-1,3-diol (synonym: resveratrol; 3,4′,5 trihydroxy trans stilbene) in 5 ml of dry THF at 25° C. is added 3 mmol of SOCl(NO.sub.3) or SO(NO.sub.3).sub.2. After 1 hr, Et.sub.2O (diethyl ether) is added and the solution is washed with water, dried and evaporated. The fully nitrated product (1,3-BIS-nitrooxy-5-[(3)-2-(4-nitrooxy-phenyl)-vinyl)-benzene) and the partially nitrated products (wherein any of the hydroxyl groups are independently replaced by ONO.sub.2 groups) are purified and isolated by chromatography on silica gel.

### EXAMPLE 2

### Preparation of Piceatannol Tetranitrate

To a solution of 1 mmol of 1,2-benzenediol, 4-(2-(3,5-dihydroxyphenyl)ethenyl)-(E)-(synonym: piceatannol) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product (piceatannol tetranitrate) and the partially nitrated products (wherein any of the hydroxyl groups are independently replaced by ONO.sub.2 groups) are purified and isolated by chromatography on silica gel.

### EXAMPLE 3

### Preparation of Butein Tetranitrate

To a solution of 1 mmol of 3,4,2′,4′-tetahydroxychalcone (synonym: butein) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product butein tetranitrate and the partially nitrated products (wherein any of the hydroxyl groups are independently replaced by ONO.sub.2 groups) are purified and isolated by chromatography on silica gel.

### EXAMPLE 4

### Preparation of Isoliquiritigenin trinitrate

To a solution of 1 mmol of 4,2′,4′-trihydroxychalcone (synonym: isoliquiritigenin) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product isoliquiritienin trinitrate and the partially nitrated products (wherein any of the hydroxyl groups are independently replaced by ONO.sub.2 groups) are purified and isolated by chromatography on silica gel.

### EXAMPLE 5

### Preparation of Fisetin Tetranitrate

To a solution of 1 mmol of 3,7,3′,4′-tetrahydroxyflavone (synonym: fisetin) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product fisetin tetranitrate and the partially nitrated products (wherein any of the hydroxyl groups are independently replaced by ONO.sub.2 groups) are purified and isolated by chromatography on silica gel.

### EXAMPLE 6

### Preparation of Quercetin Pentanitrate

To a solution of 1 mmol of 3,5,7,3′,4′-pentahydroxyflavone (synonym: quercetin) in 5 ml of dry THF at 25° C. is added 5 mmol of SOCl(NO.SUB.3) or SO(NO.SUB.3).sub.2. After 1 hr, Et.sub.2O (diethyl ether) is added and the solution is washed with water, dried and evaporated. The fully nitrated product quercetin pentanitrate and the partially nitrated products (wherein any of the hydroxyl groups are independently replaced by ONO.sub.2 groups) we purified and isolated by chromatography on silica gal.

### EXAMPLE 7

### Preparation of N-(3,5-Bis-nitrooxy-phenyl)-N′-(4-nitrooxy-phenyl)-hydrazine

To a solution of 1 mmol of 5-[N′-(4-hydroxy-phenyl)-hydrazino]-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product N-(3,5-Bis-nitrooxy-phenyl)-N′-(4-nitrooxy-phenyl)-hydrazine and the partially nitrated products (wherein any of the hydroxyl groups are independently replaced by ONO.sub.2 groups) are purified and isolated by chromatography on silica gel.

### EXAMPLE 8

### Preparation of 1,3-bis-nitrooxy-5-(4-nitrooxy-phenyldisulfanyl)-benzene

To a solution of 1 mmol of 5-(4-hydroxy-phenyldisulfanyl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is wadded with water, dried and evaporated. The fully nitrated product 1,3-bis-nitrooxy-5-(4-nitrooxy-phenyldisulfanyl)-benzene and the partially nitrated products (wherein any of the hydroxyl groups are independently replaced by ONO.sub.2 groups) are purified and isolated by chromatography on silica gel.

### EXAMPLE 9

### Preparation of 1,3-bis-nitrooxy-5-(4-nitrooxy-phenylperoxy)-benzene

To a solution of 1 mmol of 5-(4-hydroxy-phenylperoxy)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,3-bis-nitrooxy-5-(4-nitrooxy-phenylperoxy)-benzene and the partially nitrated products (wherein any of the hydroxyl groups are independently replaced by ONO.sub.2 groups) are purified and isolated by chromatography on silica gel.

### EXAMPLE 10

### Preparation of 1,3-bis-nitrooxy-5-(4-nitrooxy-phenylsulfanylmethyl)-benzene

To a solution of 1 mmol of 5-(4-(hydroxy-phenylsulfanylmethyl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl other) is added and the solution is washed with water, dried and evaporated. The fully nitrated product 1,3-bis-nitrooxy-5-(4-nitrooxy-phenylsulfanylmethyl)-benzene and the partially nitrated products (wherein any of the hydroxyl groups are independently replaced by ONO.sub.2 groups) are purified and isolated by chromatography on silica gel.

### EXAMPLE 11

### Preparation of N-(3,5-bis-nitrooxy-phenyl-O-(4-nitrooxy-phenyl)-hydroxylamine

To a solution of 1 mmol of 5-(4-hydroxy-phenoxyamino)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product N-(3,5-bis-nitrooxy-phenyl-O-(4-nitrooxy-phenyl)-hydroxylamine and the partially nitrated products (wherein any of the hydroxyl groups are independently replaced by ONO.sub.2 groups) are purified and isolated by chromatography on silica gel.

### EXAMPLE 12

### Preparation of benzyl-(4-nitrooxy-phenyl)-amine

To a solution of 1 mmol of 4-benzylamino-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product benzyl-(4-nitrooxy-phenyl)-amine is purified and isolated by chromatography on silica gel.

### EXAMPLE 13

### Preparation of 2-(salicylideneamino)phenol dinitrate

To a solution of 1 mmol of 2-(salicylideneamino)phenol in 5 ml of dry THF at 25° C. is added 2 mmol of SOCl(NO.SUB.3) or SO(NO.SUB.3).sub.2. After 1 hr. Et.sub.2O (diethyl ether) is added and the solution is washed with water, dried and evaporated. The fully nitrated product 2-(salicylideneamino)phenol dinitrate and the partially nitrated products (wherein either of the hydroxyl groups are independently replaced by ONO.sub.2 groups) are purified and isolated by chromatography on silica gel.

### EXAMPLE 14

### Preparation of (2,4-bis-nitrooxy-phenyl)-(2-nitrooxy-phenyl)-diazene

To a solution of 1 mmol of 4-(2-hydroxy-phenylazo)-benzene-1,3-diol (synonym: 1,3-benzenediol, 4-((2-hydroxyphenyl)azo)-) in 5 ml of dry THY at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2,4-bis-nitroxy-phenyl)-(2-nitrooxy-phenyl)-diazene and the partially nitrated products (wherein any of the hydroxyl groups are independently replaced by ONO.sub.2 groups) are purified and isolated by chromatography on silica gel.

### EXAMPLE 15

### Preparation of bis-(2,2′-nitrooxy-phenyl)-diazene

To a solution of 1 mmol of bis-(2,2′-hydroxy-phenyl)-diazene (synonym: 1-hydroxy-2-(2-hydroxyphenylazo)benzene) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product bis-(2,2′-nitrooxy-phenyl)-diazene and the partially nitrated products (wherein either of the hydroxyl groups are independently replaced by ONO.sub.2 groups) are purified and isolated by chromatography on silica gel.

### EXAMPLE 16

### Preparation of N-(3-nitrooxy-phenyl)-benzenesulfonamide

To a solution of 1 mmol of N-(3-hydroxy-phenyl)-benzenesulfonamide (synonym: N-(3-hydroxyphenyl)benzene sulphonamide) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product N-(3-nitrooxy-phenyl)-benzenesulfonamide is purified and isolated by chromatography on silica gel.

### EXAMPLE 17

### Preparation of N-(4-nitrooxy-phenyl)-benzenesulfonamide

To a solution of 1 mmol of N-(4-hydroxy-phenyl)-benzenesulfonamide (synonym: N-(4-hydroxyphenyl)benzene sulphonamide) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product N-(4-nitrooxy-phenyl)-benzenesulfonamide is purified and isolated by chromatography on silica gel.

### EXAMPLE 18

### Preparation of 3,3′,4,5′-tetranitrooxybibenzyl

To a solution of 1 mmol of 3,3′,4,5′-tetrahydroxybibenzyl in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 3,3′,4,5′-tetranitrooxybibenzyl and the partially nitrated products (wherein any of the hydroxyl groups are independently replaced by ONO.sub.2 groups) are purified and isolated by chromatography on silica gel.

### EXAMPLE 19

### Preparation of 1-benzyloxy-2-nitrooxy-benzene

To a solution of 1 mmol of 2-benzyloxy-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-benzyloxy-2-nitrooxy-benzene is purified and isolated by chromatography on silica gel.

### EXAMPLE 20

### Preparation of benzoic acid 3-nitrooxy-phenyl ester

To a solution of 1 mmol of benzoic acid 3-hydroxy-phenyl ester (synonym: resorcinol monobenzoate) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product benzoic acid 3-nitrooxy-phenyl ester is purified and isolated by chromatography on silica get.

### EXAMPLE 21

### Preparation of 2-nitrooxy-benzoic acid phenyl ester

To a solution of 1 mmol of 2-hydroxy-benzoic acid phenyl ester (synonym: phenyl salicylate) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 2-nitrooxy-benzoic acid phenyl ester is purified and isolated by chromatography on silica gel.

### EXAMPLE 22

### Preparation of 2-nitrooxy-N-(4-nitrooxy-phenyl)-benzamide

To a solution of 1 mmol of 2-hydroxy-N-(4-hydroxy-phenyl)-benzamide (synonym: Osalmid) in 5 ml of dry THF at 25° C. is added 2 mmol of SOCl(NO.SUB.3) or SO(NO.SUB.3).sub.2. After 1 hr, Et-sub.2O (diethyl ether) is added and the solution is washed with water, dried and evaporated. The fully nitrated product 2-nitrooxy-N-(4-nitrooxy-phenyl)-benzamide and the partially nitrated products (wherein either of the hydroxyl groups are independently replaced by ONO.sub.2 groups) are purified and isolated by chromatography on silica gel.

### EXAMPLE 23

### Preparation of 2-nitrooxy-N-(3-nitrooxy-phenyl)-benzamide

To a solution of 1 mmol of 2-hydroxy-N-(3-hydroxy-phenyl)-benzamide in 5 ml of dry THF at 25° C. is added 2 mmol of SOCl(NO.SUB.3) or SO(NO.SUB.3).sub.2. After 1 hr. Et-sub.2O (diethyl ether) is added and the solution is washed with water, dried and evaporated. The fully nitrated product 2-nitrooxy-N-(3-nitrooxy-phenyl)-benzamide and the partially nitrated products (wherein either of the hydroxyl groups are independently replaced by ONO.sub.2 groups) are purified and isolated by chromatography on silica gel.

### EXAMPLE 24

### Preparation of 3,4,5-tris-nitrooxy-N-phenyl-benzamide

To a solution of 1 mmol of 3,4,5-trihydroxy-N-((Z)-1-methylene-but-2-enyl)-benzamide (synonym: gallanilide) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 3,4,5-tris-nitrooxy-N-phenyl-benzamide and the partially nitrated products (wherein any of the hydroxyl groups are independently replaced by ONO.sub.2 groups) are purified and isolated by chromatography on silica gel.

### EXAMPLE 25

### Preparation of 1-(2,4-bis-nitrooxy-phenyl)-2-phenyl-ethanone

To a solution of 1 mmol of 1-(2,4-hydroxy-phenyl)-2-phenyl-ethanone (synonym: benzyl 2,4-dihydroxyphenyl ketone) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1-(2,4-bis-nitrooxy-phenyl)-2-phenyl-ethanone and the partially nitrated products (wherein either of the hydroxyl groups are independently replaced by ONO.sub.2 groups) are purified and isolated by chromatography on silica gel.

### EXAMPLE 26

### Preparation of 1,2-bis-nitrooxy-3-phenoxy-benzene

To a solution of 1 mmol of 3-phenoxy-benzene-1,2-diol in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,2-bis-nitrooxy-3-phenoxy-benzene and the partially nitrated products (wherein either of the hydroxyl groups are independently replaced by ONO.sub.2 groups) are purified and isolated by chromatography on silica gel.

### EXAMPLE 27

### Preparation of 1,2-bis-nitrooxy-3-(2-nitrooxy-phenoxy)-benzene

To a solution of 1 mmol of 3-(2-hydroxy-phenoxy)-benzene-1,2-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,2-bis-nitrooxy-3-(2-nitrooxy-phenoxy)-benzene and the partially nitrated products (wherein any of the hydroxyl groups are independently replaced by ONO.sub.2 groups) are purified and isolated by chromatography on silica gel.

### EXAMPLE 28

### Preparation of 1-nitrooxy-2-phenoxy-benzene

To a solution of 1 mmol of 2-phenoxy-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-nitrooxy-2-phenoxy-benzene is purified and isolated by chromatography on silica gel.

### EXAMPLE 29

### Preparation of 5,5 sulphinyl bis resorcinol tetranitrate

To a solution of 1 mmol of 5,5 sulphinyl bis resorcinol in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,5 sulphinyl bis resorcinol tetranitrate and the partially nitrated products (wherein any of the hydroxyl groups are independently replaced by ONO.sub.2 groups) are purified and isolated by chromatography on silica gel.

### EXAMPLE 30

### Preparation of 1,3-benzenediol 4,4′-thiobis tetranitrate

To a solution of 1 mmol of 1,3-benzenediol 4,4′-thiobis in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,3-benzenediol 4,4′-thiobis tetranitrate and the partially nitrated products (wherein any of the hydroxyl groups are independently replaced by ONO.sub.2 groups) are purified and isolated by chromatography on silica gel.

### EXAMPLE 31

### Preparation of phenol 2,2′ thiobis dinitrate

To a solution of 1 mmol of phenol 2,2′ thiobis in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated, The fully nitrated product phenol 2,2′ thiobis dinitrate and the partially nitrated products (wherein either of the hydroxyl groups are independently replaced by ONO.sub.2 groups) are purified and isolated by chromatography on silica gel.

### EXAMPLE 32

### Preparation of 1-benzyl-2,4-bis-nitrooxy-benzene

To a solution of 1 mmol of 4-benzyl-benzene-1,3-diol (synonym: 1,3 benzenediol 3-phenyl methyl) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1-benzyl-2,4-bis-nitrooxy-benzene and the partially nitrated products (wherein either of the hydroxyl groups are independently replaced by ONO.sub.2 groups) are purified and isolated by chromatography on silica gel.

### EXAMPLE 33

### Preparation of 2-benzyl-1,4-bis-nitrooxy-benzene

To a solution of 1 mmol of 2-benzyl-benzene-1,4-diol (synonym: 1,4 benzenediol 4-phenyl methyl) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-benzyl-1,4-bis-nitrooxy-benzene and the partially nitrated products (wherein either of the hydroxyl groups are independently replaced by ONO.sub.2 groups) are purified and isolated by chromatography on silica gel.

### EXAMPLE 34

### Preparation of (2,3,4-tris-nitrooxy-phenyl)-(3,4,5-tris-nitrooxy-phenyl)-methanone

To a solution of 1 mmol of (2,3,4-trihydrooxy-phenyl)-(3,4,5-trihydroxy-phenyl)-methanone (synonym: Exifone) in 5 ml of dry THF at 25° C. is added 6 mmol of SOCl(NO.SUB.3) or SO(NO.SUB.3).sub.2. After 1 hr, Et.sub.2O (diethyl ether) is added and the solution is washed with water, dried and evaporated. The fully nitrated product (2,3,4-tris-nitrooxy-phenyl)-(3,4,5-tris-nitrooxy-phenyl)-methanone and the partially nitrated products (wherein any of the hydroxyl groups are independently replaced by ONO.sub.2 groups) are purified and isolated by chromatography on silica gel.

### EXAMPLE 35

### Preparation of (2-nitrooxy-phenyl)-phenyl-amine

To a solution of 1 mmol of 2-phenylamino-phenol in 5 ml of dry THF at 25° C. is added 1 mmol of SOCl(NO.SUB.3) or SO(NO.SUB.3).sub2. After 1 hr, Et.sub.2O (diethyl ether) is added and the solution is washed with water, dried and evaporated. The nitrated product (2-nitrooxy-phenyl)-phenyl-amine is purified and isolated by chromatography on silica gel.

### EXAMPLE 36

### Preparation of 2-(3,5-bis-nitrooxy-phenyl)-6-nitrooxy-4H-chromene

To a solution of 1 mmol of 5-(6-hydroxy-4H-chromen-2-yl-benzene-1,3-diol in 5 ml of dry THF at 25° C. is added 3 mmol of SOCl(NO.SUB.3) or SO(NO.SUB.3).sub.2. After 1 hr. Et.sub.2O (diethyl ether) is added and the solution is washed with water, died and evaporated. The fully nitrated product 2-(3,5-bis-nitrooxy-phenyl)-6-nitrooxy-4H-chromene and the partially nitrated products (wherein any of the hydroxyl groups are independently replaced by ONO.sub.2 groups) are purified and isolated by chromatography on silica gel.

### EXAMPLE 37

### Preparation of 2-(3,5-bis-nitrooxy-phenyl)-6-nitrooxy-1,4-dihydro-naphthalene

To a solution of 1 mmol of 5-(6-hydroxy-1,4-dihydro-naphthalen-2yl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,5-bis-nitrooxy-phenyl)-6-nitrooxy-1,4-dihydro-naphthalene and the partially nitrated products (wherein any of the hydroxyl groups are independently replaced by ONO.sub.2 groups) are purified and isolated by chromatography on silica gel.

### EXAMPLE 38

### Preparation of 2-(3,5-bis-nitrooxy-phenyl)-6-nitrooxy-1,2,3,4-tetrahydro-naphthalene

To a solution of 1 mmol of 5-(6-hydroxy-1,2,3,4-tetrahydro-naphthalen-2-yl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,4-bis-nitrooxy-phenyl)-6-nitrooxy-1,2,3,4-tetrahydro-naphthalene and the partially nitrated products (wherein any of the hydroxyl groups are independently replaced by ONO.sub.2 groups) are purified and isolated by chromatography on silica gel.

### EXAMPLE 39

### Preparation of 5,7-bis-nitrooxy-2-(4-nitrooxy-phenyl)-chroman-4-one

To a solution of 1 mmol of 5,7-dihydroxy-2-(4-hydroxy-phenyl)-chroman-4-one (Synonym: naringenin) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,7-bis-nitrooxy-2-(4-nitrooxy-phenyl)-chroman-4-one and the partially nitrated products (wherein any of the hydroxyl groups are independently replaced by ONO.sub.2 groups) are purified and isolated by chromatography on silica gel.

### EXAMPLE 40

### Preparation of 5,7-bis-nitrooxy-2-(4-nitrooxy-phenyl)-chromen-4-one

To a solution of 1 mmol of 5,7-dihydroxy-2-(4-hydroxy-phenyl)-chromen-4-one (Synonym: apigenin) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,7-bis-nitrooxy-2-(4-nitrooxy-phenyl)-chromen-4-one and the partially nitrated products (wherein any of the hydroxyl groups are independently replaced by ONO.sub.2 groups) are purified and isolated by chromatography on silica gel.

### EXAMPLE 41

### Preparation of 5,7-bis-nitrooxy-3-(4-nitrooxy-phenyl)-chromen-4-one

To a solution of 1 mmol of 5,7-dihydroxy-3-(4-hydroxy-phenyl)-chromen-4-one (Synonym: genistein) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,7-bis-nitrooxy-3-(4-nitrooxy-phenyl)-chromen-4-one and the partially nitrated products (wherein any of the hydroxyl groups are independently replaced by ONO.sub.2 groups) are purified and isolated by chromatography on silica gel.

### EXAMPLE 42

### Preparation of 2-(3,4-bis-nitrooxy-phenyl)-3,4,5,7-tetrakis-nitrooxy-chroman

To a solution of 1 mmol of 2-(3,4-dihydroxy-phenyl)-chroman-3,4,5,7-tetraol (synonym: leucocianidol) in 5 ml of dry THF at 25° C. is added 6 mmol of SOCl(NO.SUB.3) or SO(NO.SUB.3).sub.2. After 1 hr, Et.sub.2O (diethyl ether) is added and the solution is washed with waters, dried and evaporated. The fully nitrated product 2-(3,4-bis-nitrooxy-phenyl)-3,4,5,7-tetrakis-nitrooxy-chroman and the partially nitrated products (wherein any of the hydroxyl groups are independently replaced by ONO.sub.2 groups) are purified and isolated by chromatography on silica gel.

### EXAMPLE 43

### Preparation of 6-hydroxy-7-nitrooxy-3-(4-nitrooxy-phenyl)-chroman-4-one

To a solution of 1 mmol of 6,7-dihydroxy-3-(4-hydroxy-phenyl)-chroman-4-one (Synonym: 6,7,4′-trihydroxyisoflavanone) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 6-hydroxy-7-nitrooxy-3-(4-nitrooxy-phenyl)-chroman-4-one and the partially nitrated products (wherein any of the hydroxyl groups are independently replaced by ONO.sub.2 groups) are purified and isolated by chromatography on silica gel.

### EXAMPLE 44

### Preparation of Quracol B tetranitrate

To a solution of 1 mmol of Quracol B in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product Quracol B tetranitrate and the partially nitrated products (wherein any of the hydroxyl groups are independently replaced by ONO.sub.2 groups) are purified and isolated by chromatography on silica gel.

### EXAMPLE 45

### Preparation of 1-(4-hydroxy-2,6-bis-nitrooxy-phenyl)-3-(4-nitrooxy-phenyl)-propan-1-one

To a solution of 1 mmol of 3-(4-hydroxy-phenyl)-1-(2,4,6-trihydroxy-phenyl)-propan-1-one (Synonym: phloretin) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1-(4-hydroxy-2,6-bis-nitrooxy-phenyl)-3-(4-nitrooxy-phenyl)-propan-1-one and the partially nitrated products (wherein any of the hydroxyl groups are independently replaced by ONO.sub.2 groups) are purified and isolated by chromatography on silica gel.

### EXAMPLE 46

### Preparation of 1-nitrooxy-4-((Z)-3-phenyl-allyl)-benzene

To a solution of 1 mmol of 4-((Z)-3-phenyl-allyl)-phenol (synonym: 4(-3-phenyl-2-propenyl)-,(E)-phenol) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-nitrooxy-4-((Z)-3-phenyl-allyl)-benzene is purified and isolated by chromatography on silica gel.

### EXAMPLE 47

### Preparation of 1-nitrooxy-4-((E)-3-phenyl-propenyl)-benzene

To a solution of 1 mmol of 4-((E)-3-phenyl-propenyl)-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-nitrooxy-4-((E)-3-phenyl-propenyl)-benzene is purified and isolated by chromatography on silica gel.

### EXAMPLE 48

### Preparation of 5,6,7-tris-nitrooxy-2-phenyl-chromen-4-one

To a solution of 1 mmol of 5,6,7-trihydroxy-2-phenyl-chromen-4-one (synonym: baicalein) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,6,7-tris-nitrooxy-2-phenyl-chromen-4-one and the partially nitrated products (wherein any of the hydroxyl groups are independently replaced by ONO.sub.2 groups) are purified and isolated by chromatography on silica gel.

### EXAMPLE 49

### Preparation of Rutin Tetranitrate

To a solution of 1 mmol of 2-(3,4-dihydroxy-phenyl)-5,7-dihydroxy-3-[(2S,3R,5S,6R)-3,4,5-trihydroxy-6-((2R,3R,4R,5R,6S)-3,4,5-trihydroxy-6-methyl-tetrahydro-pyran-2-yloxymethyl)-tetrahydro-pyran-2-yloxy]-chromen-4-one (Synonym: rutin) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,4-bis-nitrooxy-phenyl)-5,7-bis-nitrooxy-3-[(2S,3R,5S,6R)-3,4,5-trihydroxy-6-((2R,3R,4R,5R,6S)-3,4,5-trihydroxy-6-methyl-tetrahydro-pyran-2-yloxymethyl)-tetrahydro-pyran-2-yloxy]-chromen-4-one (rutin tetranitrate) and the partially nitrated products (wherein any of the hydroxyl groups are independently replaced by ONO.sub.2 groups) are purified and isolated by chromatography on silica gel.

### EXAMPLE 50

### Preparation of 5-hydroxy-2-(4-hydroxyphenyl)-7-(2-O-alpha-L-rhamnopyranosyl-beta-D-glucopyranosyloxy)-4-chromanon dinitrate

To a solution of 1 mmol of 5-hydroxy-2-(4-hydroxyphenyl)-7-(2-O-alpha-L-rhamnopyranosyl-beta-D-glucopyranosyloxy)-4-chromanon (synonym: naringin) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5-hydroxy-2-(4-hydroxyphenyl)-7-(2-O-alpha-L-rhamnopyranosyl-beta-D-glucopyranosyloxy)-4-chromanon dinitrate and the partially nitrated products (wherein either of the hydroxyl groups are independently replaced by ONO.sub.2 groups) are purified and isolated by chromatography on silica gel.

### EXAMPLE 51

### Preparation of (E)-(3S,5R)-7-[3-(4-fluoro-phenyl)-1-isopropyl-1H-indol-2-yl]-1,3,5-tris-nitrooxy-hept-6-en-1-one

To a solution of 1 mmol of (E)-(3S,5R)-7-[3-(4-fluoro-phenyl)-1-isopropyl-1H-indol-2-yl]-3,5-dihydroxy-hept-6-enoic acid (Synonym: fluvastatin; Novartis) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product (E)-(3S,5R)-7-[3-(4-fluoro-phenyl)-1-isopropyl-1H-indol-2-yl]-1,3,5-tris-nitrooxy-hept-6-en-1-one and the partially nitrated products (wherein any of the hydroxyl groups are independently replaced by ONO.sub.2 groups) are purified and isolated by chromatography on silica gel.

### EXAMPLE 52

### Preparation of 5-(4-fluoro-phenyl)-2-isopropyl-4-phenyl-1-((3R,5R)-3,5,7-tris-nitrooxy-7-oxo-heptyl)-1H-pyrrol-1-yl]-3-carboxylic acid phenylamide

To a solution of 1 mmol of (3R,5R)-7-[2-(4-fluoro-phenyl)-5-isopropyl-3-phenyl-4-phenylcarbamoyl-pyrrol-1-yl]-3,5-dihydroxy-heptanoic acid (Synonym: atorvastatin; Parke-Davis) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5-(4-fluoro-phenyl)-2-isopropyl-4-phenyl-1-((3R,5R)-3,5,7-tris-nitrooxy-7-oxo-heptyl)-1H-pyrrol-1-yl]-3-carboxylic acid phenylamide and the partially nitrated products (wherein any of the hydroxyl groups are independently replaced by ONO.sub.2 groups) are purified and isolated by chromatography on silica gel.

### EXAMPLE 53

### Preparation of (E)-(3R,5S)-7-[4-(4-fluoro-phenyl)-2,6-diisopropyl-5-methoxymethyl-pyridin-3-yl]-1,3,5-tris-nitrooxy-hept-6-en-1-one

To a solution of 1 mmol of (E)-(3R,5S)-7-[4-(4-fluoro-phenyl)-2,6-diisopropyl-5-methoxymethyl-pyridin-3-yl]-3,5-dihydroxy-hept-6-enoic acid (Synonym: cerivastatin; Bayer) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product (E)-(3R,5S)-7-[4-(4-fluoro-phenyl)-2,6-diisopropyl-5-methoxymethyl-pyridin-3-yl]-1,3,5-tri-nitrooxy-hept-6-en-1-one and the partially nitrated products (wherein any of the hydroxyl groups are independently replaced by ONO.sub.2 groups) are purified and isolated by chromatography on silica gel.

### EXAMPLE 54

### Preparation of (S)-2-methyl-butyric acid (1S,3S,7S,8S,8aR)-7-methyl-3-nitrooxy-8-((4R,6R)-3,5,7-tris-nitrooxy-7-oxo-heptyl)-1,2,3,7,8,8a-hexahydro-napthaten-1-yl ester

To a solution of 1 mmol of (2R,4R)-3,5-dihydroxy-7-[(1S,2S,6S,8S,8aR)-6-hydroxy-2-methyl-8-((S)-2-methyl-butyryloxy)-1,2,6,7,8,8a-hexahydro-napthalen-1-yl]-heptanoic acid (Synonym: pravastatin; Bristol-Myers Squibb) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product (S)-2-methyl-butyric acid (1S,3S,7S,8S,8aR)-7-methyl-3-nitrooxy-8-((4R,6R)-3,5,7-tis-nitrooxy-7-oxo-heptyl)-1,2,3,7,8,8a-hexahydro-naphthalen-1-yl ester and the partially nitrated products (wherein any of the hydroxyl groups are independently replaced by ONO.sub.2 groups) are purified and isolated by chromatography on silica gel.

### EXAMPLE 55

### Preparation of 2,2-dimethyl-butyric acid (1S,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester

To a solution of 1 mmol of 2,2-dimethyl-butyric acid (1S,3R,7S,8S,8aR)-8-[2-((2R,4R)-4-hydroxy-6-oxo-tetrahydro-pyran-2-yl)-ethyl]-3,7-dimethyl-1,2,3,7,8,8a-hexahydro-napthalen-1-yl ester (synonym: simvastatin; Merck) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 2,2-dimethyl-butyric acid (1S,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester is purified and isolated by chromatography on silica gel.

### EXAMPLE 56

### Preparation of (S)-2-methyl-butyric acid (1S,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester

To a solution of 1 mmol of (S)-2-methyl-butyric acid (1S,3R,7S,8S,8aR)-8-[2-((2R,4R)-4-hydroxy-6-oxo-tetrahydro-pyran-2-yl)-ethyl]-3,7-dimethyl-1,2,3,7,8,8a-hexahydro-naphthalen-1-yl ester (synonym: lovastatin; Merck) in 5 ml of dry THF at 25° C. is added 1 mmol of SOCl(NO.SUB.3) or SO(NO.SUB.3).sub.2, After 1 hr, Et.sub.2O (diethyl ether) is added and the solution is washed with water, dried and evaporated. The nitrated product (S)-2-methyl-butyric acid (1S,3R,7S,8S,8aR)-3,7-dimethyl-[2-((2R,4R)-4-nitrooxy-6-oxo-tetrahydro-pyran-2-yl)-ethyl]1,2,3,7,8,8a-hexahydro-napthalen-1-yl ester is purified and isolated by chromatography on silica gel.

### EXAMPLE 57

### Preparation of N-[4-(4-fluoro-phenyl)-6-isopropyl-5-((e)-(3R,5R)-3,5,7-tris-nitrooxy-7-oxo-hept-1-enyl)-pyrimidin-2-yl]-N-methyl-methanesulfonamide

To a solution of 1 mmol of (E)-(3,5R)-7-[4-(4-fluoro-phenyl)-6-isopropyl-2-(methanesulfonyl-methyl-amino)-pyrimidin-5-yl]-3,5-dihydroxy-hept-6-enoic acid (synonym, rosuvastatin; Astra-Zeneca) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product N-[4-(4-fluoro-phenyl)-6-isopropyl-5-((E)-(3R,5R)-3,5,7-tris-nitroxy-7-oxo-hept-1-enyl)-pyrimidin-2-yl]-N-methyl-methanesulfonamide is purified and isolated by chromatography on silica gel.

### EXAMPLE 58

### Preparation of Nitrooxy-pyridin-3-yl-methanone

To a solution of 1 mmol of nicotinic acid (synonym: niacin) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product nitrooxy-pyridin-3-yl-methanone is purified and isolated by chromatography on silica gel.

### EXAMPLE 59

### Preparation of (S)-1-(4-fluoro-phenyl)-3-[(S)-3-(4-fluoro-phenyl)-3-nitrooxy-propyl]-4-(4-nitrooxy-phenyl)-azetidin-2-one

To a solution of 1 mmol of (S)-1-(4-fluoro-phenyl)-3-[(S)-3-(4-fluoro-phenyl)-3-hydroxy-propyl]-4-(4-hydroxy-phenyl)-azetidin-2-one (synonym: ezetimibe; Merck) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product (S)-1-(4-fluoro-phenyl)-3-[(S)-3-(4-fluoro-phenyl)-3-nitrooxy-propyl]-4-(4-nitrooxy-phenyl)-azetidin-2-one and the partially nitrated products (wherein either of the hydroxyl groups are independently replaced by ONO.sub.2 groups) are purified and isolated by chromatography on silica gel.

### EXAMPLE 60

### Method for Glucoronidating Compounds of the Invention

This example describes the method of preparing glucoronidated compounds of the invention. In this specific example, a dinitrated version of resveratrol, 3,4′-nitrooxy-5-hydroxy resveratrol (50-1000 μM) prepared as in Example 1 and 10 μl of human intestinal, 25 μl of colon or 10 μl of liver microsomes (200, 400, 200 μg of protein, respectively), 20 of μl recombinant UDP-glucuronosyltransferase (400 μg of protein), in a final volume of 500 μl of 50 mM Tris HCl buffer (pH 7.8) with 10 mM MgCl₂ are preincubated for 5 min at 37° C., The reactions are initiated by the addition of 1 mM 5′-diphosphoglucuronic acid. The reaction mixtures are incubated at 37° C. for 60 min. The samples are cooled on ice and subjected to solid-phase extraction using oasis Hydrophilic-Lipophilic Balance 1 cc C₁₈ extraction cartridges (Waters Corp, Milford, Mass.). The cartridges are washed with 1-ml methanol and equilibrated with 1-ml water. After loading 0.5 ml of the sample, the cartridges are washed with 5% methanol and eluted with 2 ml of 100% methanol. The methanol eluate is dried under N₂ gas at 40° C., and the sample is redissolved in 250 μl of mobile phase for HPLC analysis.

### EXAMPLE 61

### Method for Sulfating Compounds of the Invention

This example describes the method of preparing sulfated compounds of the invention. In this specific example, a dinitrated version of resveratrol, 3,4′-nitrooxy-5-hydroxy resveratrol prepared as in Example 1 is sulfated by a sulfotransferase enzyme using a previously described ion-pair extraction method (Varin et al. 1987. Anal. Biochem. 161:176-180). The typical reaction mixture contains 0.1 to 200 μM of 3,4′-nitrooxy-5-hydroxy resveratrol, 1 μM [³⁵S]PAPS and 2.5 μl of pooled human liver cytosol (50 μg of protein), 2.5 μl of human jejunal cytosol (30 μg), Caco-2 cytosol (225 μg) or 0.25 μl recombinant sulfotransferase in 33 mM Tris-HCl buffer, pH 7.4, with 8 mM dithiothreitol and 0.0625% bovine serum albumin in a total volume of 100 μl. The samples are incubated for 30 min at 37° C., and the reactions terminated by the addition of 10 μl 2.5% acetic acid, 20 μl of 0.1 μM tetrabutylammonium hydrogen sulfate and 500 μl of ethyl acetate. After through mixing and centrifugation, 400 μl of the ethyl acetate extract is subjected to liquid scintillation counting after the addition of biodegradable counting scintillant (Amersham Biosciences, Piscataway, N.J.).

### EXAMPLE 62

### Resveratrol Treatment of CaCo2 Cells, from Intestine

This study determined whether resveratrol had an effect on APO A1 gene in CaCo2 cells, an intestinal cell line. Cells were grown under conditions recommended by the ATCC and summarized briefly in the methods section. The initial studies examined the potential effects of resveratrol to increase APO A1 expression using histologic analysis. Cells were treated with 5 or 10 μM of resveratrol and then stained for their abundance of APO A1 using a commercially available human APO A1 antibody (data not shown). The CaCo2 cells were examined using phase contrast and immunohistochemical staining of APO A1 protein in the absence (untreated) and presence of resveratrol (5 and 10 μM), resveratrol caused an increase in the abundance of APO A1 signal following exposure to 5 and 10 μM of the agent after 36 hours of treatment. An increase in the level of APO A1 protein expression in the presence of resveratrol was also demonstrated. The results showed that both 5 and 10 μM of resveratrol increased the fluorescence arising from cellular content of APO A1 protein.

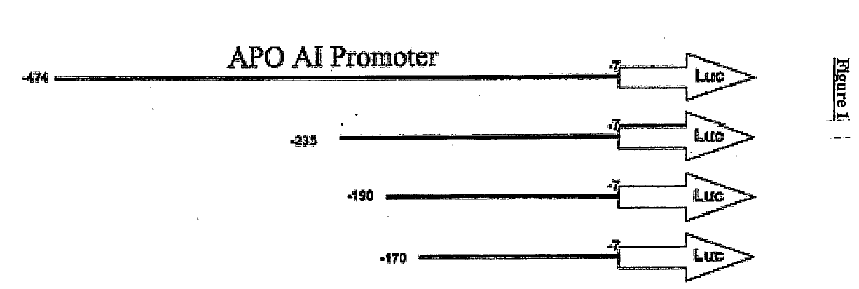

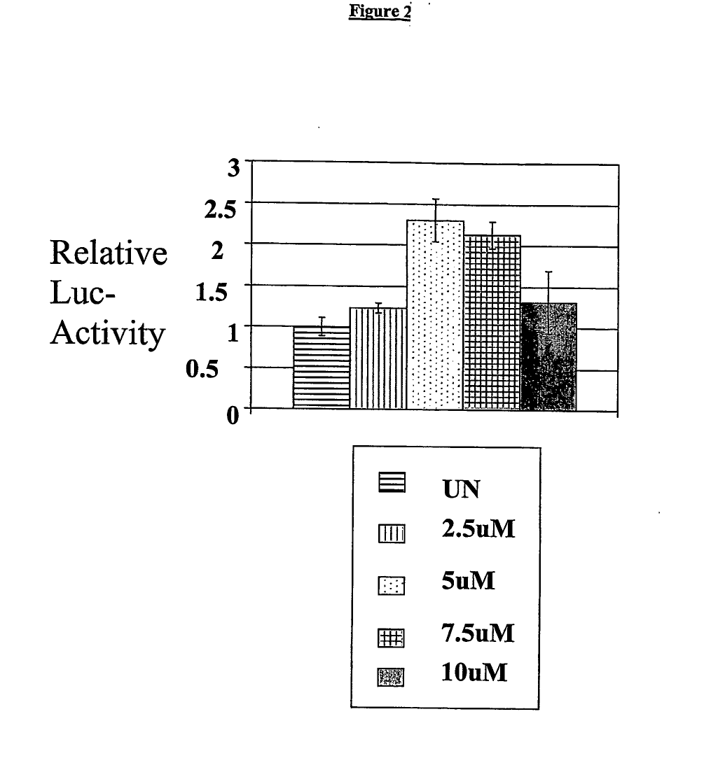

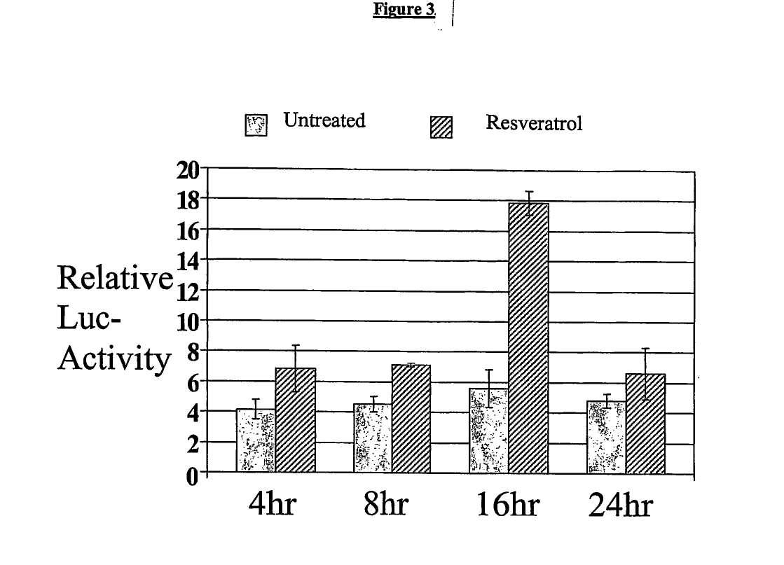

Next the CaCo2 cells were exposed to varying concentrations of resveratrol from 0 to 15 μM. The calls were transfected, using a standard technique, with the reporter construct, pA1.474-Luc (see map, FIG. 1) along with pRSV-β-galactosidase as a monitor for transfection efficiency. The pA1.474-Luc is a construct that we have created using conventional molecular biology techniques and contains the human APO A1 promoter from −474 to −7 fused to the reporter, firefly luciferase (Luc). The resveratrol was dissolved DMSO and then added to the culture media to yield a final concentration that varied from 0 to 15 μM. The cells were treated with the varying concentrations of the resveratrol for 16 hours. At the end of the treatment, the cells were harvested and the Luc-activity measured. These values were normalized to both lysate protein concentration and also 3-galactosidase activity. The results (FIG. 2) showed that the resveratrol stimulated APO A1 promoter activity maximally by 2.5-fold at a resveratrol concentration that ranged from 5 to 7.5 μM.

Whereas, the preceding studies showed that the resveratrol concentration, which caused maximal stimulation of the APO A1 promoter activity ranged between 5-7.5 μM, the duration of action was unclear. In order to address this point, the same experiment to that above was used to assess the kinetics of resveratrol induction of the APO A1 promoter. CaCo2 cells transfected with pA1.474-Luc were treated with 5 μM of resveratrol at selected time points varying from 4 to 24 hours. This construct pA1.474-Luc contained the rat APO A1 promoter DNA spanning −474 to −7 fused to the reporter gene, firefly luciferase (Luc). A significant effect was observed at 4, 8, 16 and 24 hours following administration of resveratrol but maximal stimulation appeared following 16 hours of exposure to the compound. Results (FIG. 3) showed that the optimal time point for the stimulatory effects of resveratrol on the APO A1 promoter appeared to be around 16 hours. The information arising from these studies show that resveratrol can stimulate APO A1 gene transcription in CaCo2 cells and the time of maximal effect for resveratrol is roughly 16 hours after exposure.

### EXAMPLE 63

### Effects of Resveratrol Require a Fragment of the DNA Spanning Nucleotides −190 to −170

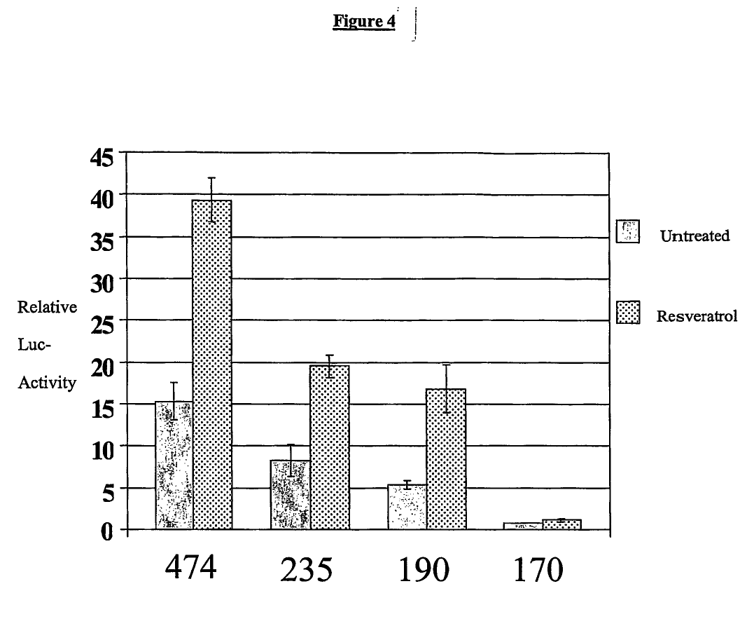

Since pA1.474-Luc, used in the above studies, was found to mediate effects of resveratrol and this construct contained the human APO A1 DNA fragment spanning −474 to −7, we postulated that a motif or motifs within this segment of the promoter DNA mediates actions of the compound. In order to identify the potential motif(s), separate constructs containing progressively smaller amounts of APO A1 DNA were fused to the Luc gene. The activity of each construct was tested by transient transfection assay in CaCo2 cells and than treated with 5 μM resveratrol for a minimum of 16 hours. Results (FIG. 4) showed that the full-length (−474 to −7) promoter produced a 2.5-fold induction. The number at the bottom of each set of columns denotes the 5′ location of the fragment and the 3′ end is common to all deletional clones at −7. For example, the left set of columns shows activity of the −474 to −7 fragment in the presence and absence of resveratrol, respectively. These results demonstrate that removal of the DNA from −190 to −171 of the promoter abolishes the response to resveratrol. Removal of the DNA the −235 or −190 to −7 fragments from the parent promoter did not affect the ability of resveratrol to induce the 2.5-fold increase in promoter activity. In contrast, further deletion with the remaining −170 to −7 fragment of the promoted abolished the resveratrol induction of the promoter. We discovered the resveratrol responsive motif in the APO A1 DNA must span nucleotides −190 to −170.

### EXAMPLE 64

### Resveratrol Increases APO A1 Protein Secreted from CaCO2 Cells

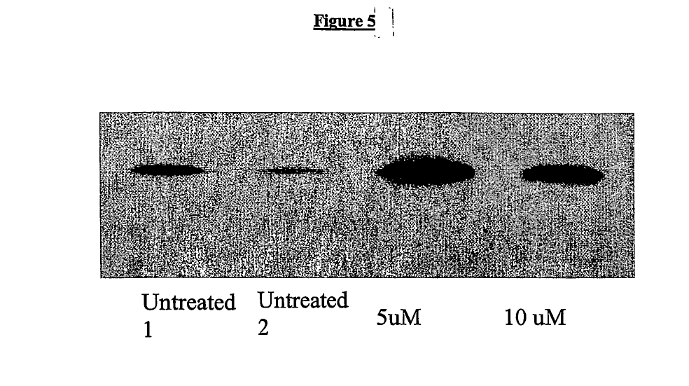

This to experiment sought to measure whether resveratrol stimulation of transcriptional activity of the promoter in the CaCo2 cells increased the abundance of the APO A1 protein, ultimately responsible for the antiatherogenic activity of the gene. Resveratrol increased activity of the APO A1 promoter in the pA1.474-Luc construct, a transgene that is introduced into CaCo2 cells by transient transfection but whether it affected activity of the APO A1 gene endogenous to the CaCo2 cells was not known. For these studies, CaCo2 cells were cultured as usual and exposed to media containing resveratrol at a concentration of 5 or 10 p,M for 36 hours. Longer exposure of the cells to resveratrol was utilized to allow adequate time for the APO A1 protein to be secreted into the media from the CaCo2 cells, and detected. Spent media exposed to the cells for 36 hours was assayed for its content of APO A1 protein using western blot analysis. Results (FIG. 5) showed a marked increase in abundance of APO A1 protein in the spent media from cells treated with resveratrol but APO A1 in the media lacking resveratrol was lower.

The results of these studies show that the antiatherogenic properties of resveratrol augments expression of the APO A1 gene. Increased expression of the APO A1 gene augments RCT and thereby enhances the removal of cholesterol from the body. The data in CaCo2 cells are significant and we have unexpectedly:
1) Identified for the first time effects of resveratrol on APO A1 in intestinal cells.
2) Identified that resveratrol affects transcription of the APO A1 gene.
3) Determined the time required for resveratrol to act on APO A1 in the cells.
4) Determined the range of resveratrol concentration to therapeutically alter APO A1 gene expression.
5) Identified the DNA motif that mediates resveratrol effects in CaCo2 cells.
6) Showed that one effect of resveratrol is to increase abundance of APO A1 protein.

This information will be useful in harnessing the of use of resveratrol or other similar APO A1 increasing agents by:
1) Designing a formulation of resveratrol that may be released into the intestine.
2) Designing a formulation for timed release of resveratrol or such agents to insure that it will be in the intestinal track for a minimum of 16 hours.
3) Designing a formulation for maintaining presence of a therapeutic dose of resveratrol or such agents that was not previously known.
4) Demonstrating use of various reporter constructs and cell lines for assaying the actions of resveratrol or such agents and extending it for screening of natural or synthetic polyphenols or other agents similar in action to that of resveratrol.

### EXAMPLE 65

### Resveratrol Treatment of Hep G2 Cells, from Liver

Since the APO A1 gene is expressed in both liver and small intestine, the following studies examine the ability of resveratrol to affect expression of the gene in liver cells. The first set of studies examined the potential ability of resveratrol to increase the abundance of APO A1 and to assess this possibility using histological analysis. Cells were grown under conditions recommended by the ATCC and summarized briefly in the methods section. The initial studies examined the potential effects of resveratrol to increase APO A1 expression using histologic analysis. Cells were treated with 5 or 10 μM of resveratrol and then stained for their abundance of APO A1 using a commercially available human APO A1 antibody. Hep G2 cells were viewed under phase contrast or fluorescence microscopy following treatment with or without resveratrol and immunostaining for their content of APO A1 protein. The results showed an increase in fluorescence for APO A1 signal following treatment with 5 or 10 μM of resveratrol.

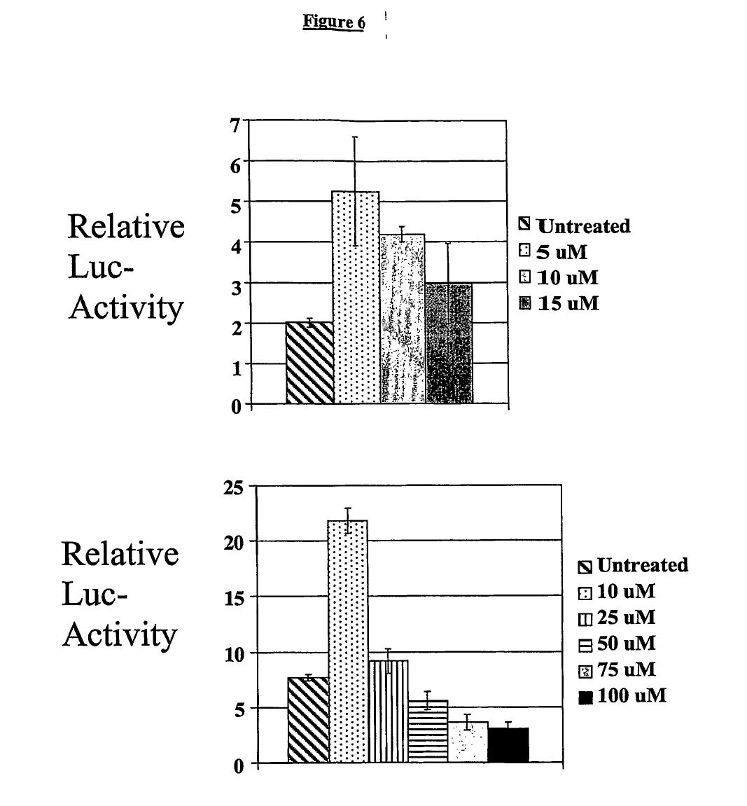

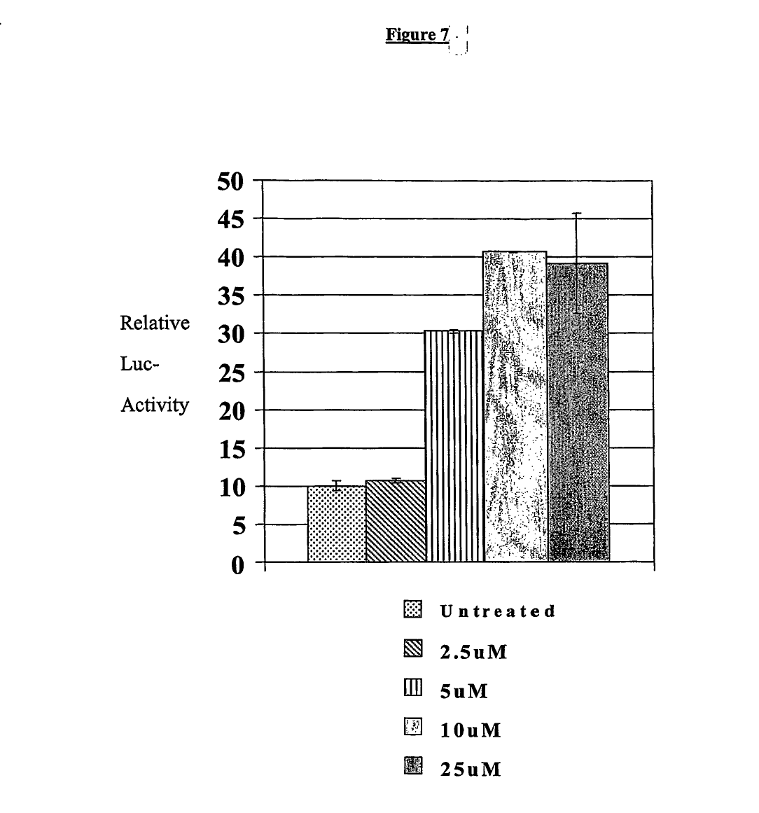

To assay for promoter activity in Hep G2 cells, the reporter construct pA1.474-Luc was inserted into the human hepatoma, Hep G2, cells along with pRSV-β-galactosidase as a monitor for transfection efficiency using conventional molecular biology techniques as later described. The transfected cells were exposed to varying concentrations of resveratrol from 0 to 100 μM for 16 hours. The cells were harvested and assayed for Luc-activity. Cells treated with 0, 5, 10, 25, 50, 75 and 100 μM resveratrol showed a dose-response relationship with peak dose at 5 to 10 μM, but becoming inhibitory at 50 μM and above. These data have been normalized to β-gal (co-transfected reporter to control for transfection efficiency) and expressed relative to the protein levels. The experiment was repeated 3 times with 3 different batches of cells The values obtained were normalized relative to both protein and 6-galactosidase activity. Results (FIG. 6) showed a 3-fold increase in activity following treatment with 5 to 10 pM resveratrol. However, further increases in the concentration of resveratrol did not further increase Luc-activity of the reporter construct and in fact, concentrations of the compound at 15, 25, 50, 75 or 100 μM were associated with no significant increases but rather led to a decrease of 50% in Luc-activity, To verify these observations, a cell line was created that contained the pA1.474-Luc permanently inserted into the cells. These permanently transfected cells were tested for response to resveratrol concentrations ranging from 0-20 μM. The cells that were neomycin resistant and had Luc-activity were retained for the studies because they contain both the pA1.474-Luc and the neomycin resistance marker. These cells were treated with resveratrol (0 to 25 μM). To create the permanently transfected cells, 474-Luc was co-transfected with another plasmid carrying neomycin resistance. The ability to grow in neomycin was a marker for successful transfection. A dose-response effect to resveratrol was observed with results mimicking that of transiently transfected cells. Results (FIG. 7) showed that Luc-activity in the permanently transfected cells increased in a dose dependent fashion with a maximal increase of 4-fold following treatment with 10 μM resveratrol.

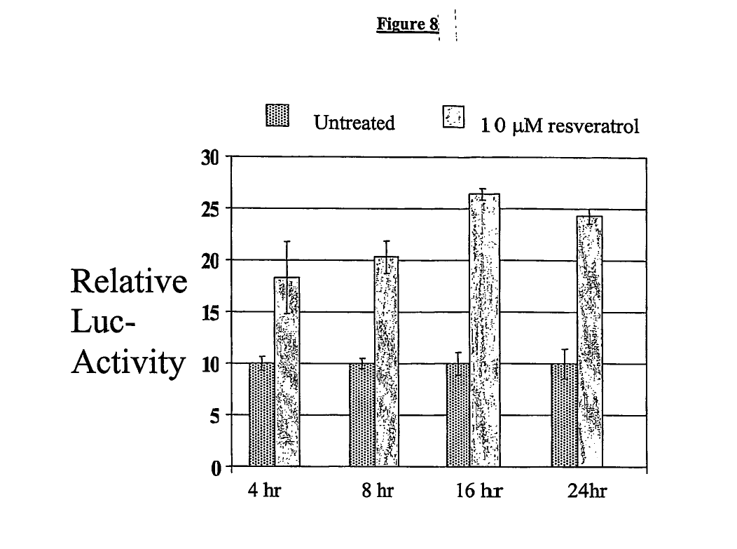

The time course of pA1.474-Luc was tested in response to a fixed concentration of resveratrol. In this study Hep G2 cells were transiently transfected with pA1.474-Luc and then exposed to 10 p.M resveratrol. The cells were harvested at 4, 8, 16 and 24 hours. The Luc-activity was assayed in the cells and results showed that maximal stimulation of the promoter began at 16 and extended to 24 hrs. The maximal effect of the resveratrol was similar to that in the CaCo2 cells with maximal increase observed after 16 hours of treatment (FIG. 8).

### EXAMPLE 66

### Resveratrol Increases APO A1 Protein Secreted from Hep G2 Cells

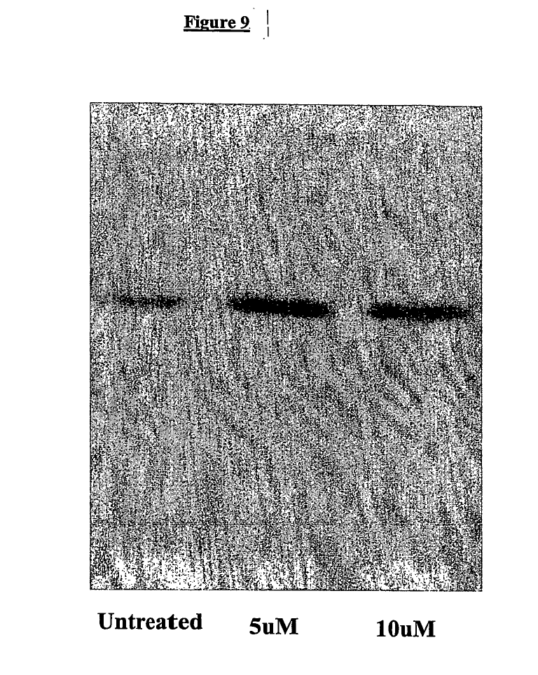

To measure whether resveratrol stimulation of the APO A1 promoter in the Hep G2 cells also increases the abundance of the protein, APO A1 secreted into the media was assessed following treatment with the compound. Resveratrol increased the activity of the APO A1 promoter in the pA1.474-Luc construct, a transgene that was introduced into Hep G2 cells by transient or sale transfection. Hep G2 cells were cultured as usual and exposed to media containing resveratrol at a concentration of 5 or 10 p,M for 36 hours. Spent media exposed to the cells for 36 hours were assayed for its content of APO A1 protein using western blot analysis. Results (FIG. 9) showed a marked increase in abundance of APO A1 protein in the spent media from cells treated with resveratrol but APO A1 in the media lacking resveratrol was lower.

These experiments demonstrate that resveratrol also unexpectedly and advantageously increased expression of the APO A1 gene in Hep G2 cells derived from liver. A preferred embodiment of a screening assay would therefore advantageously contain a permanently transfected Hep G2 cell line containing the pA1.474-marker where a preferred marker is Luc. Such cells could be used to screen for compounds or agents for increasing APO A1 expression or transfection. The experiments show the preferred time periods for therapeutic application of such compounds as well as how the preferred therapeutic concentrations may be initially determined. Of course, it will be readily recognized that conventional clinical trials are needed to refine therapeutic regimens in accordance with their purpose.

We have discovered resveratrol to advantageously affect the expression of the APO A1 gene. Using human cell lines, Hep G2 and CaCo2, an increase in levels of APO A1 protein and promoter activity in both cell types exposed to resveratrol concentrations in the range of 5-10 μM was observed. Equally important is that exposure of cells to concentrations that exceed this range has a detrimental effect on expression of the APO A1 gene. In addition, the finding that gene activity in response to a single exposure of resveratrol had maximal effect on transcription of the gene at 16-24 hours but levels of the protein could be detected up to 36 hours after exposure is also new information that guides determination of the length of time required for exposure of the cells to resveratrol for therapeutic effect. The fact that CaCo2 derived intestinal cells respond to resveratrol is also new. This fact is important because resveratrol will contact the intestinal cells first before going to the liver and therefore, the interaction and effect of resveratrol on intestinal cells is likely more important then its effect on liver cells because the concentrations of resveratrol after consumption may never reach levels in the blood to sufficiently stimulate the liver cells.

In addition to these basic observations, the mechanism by which resveratrol stimulated APO A1 gene transcription was tested in assays that employed deletional constructs of the promoter. These studies show that resveratrol in the CaCo2 cells act via the −190 to −170 fragment of DNA but the effect in liver cells may be due to interaction at the same or different site. This is important because in order to produce a beneficial effect in the intestinal cells using derivatives or analogues of resveratrol, it may be different from that on the liver.

In another embodiment of this invention, permanently transfected HepG2 cells are used as a screening system to screen for the resveratrol sensitive promotor sequence in other genes. Permanently transfected HepG2 or CaCo2 cells with deletional constructs can provide the basis of an assay system for screening of resveratrol sensitive promotor sequences in genes, and for screening neutraceuticals and pharmaceuticals to identify those that may regulate APO A1 expression.

### EXAMPLE 67

### Measurement of ApoA-1 Protein Expression

This study measures the effect of the compounds on the APO A1 gene in CaCo2 cells, an intestinal cell line, or in Hep G2 cells, a hepatoma cell line. Cells are treated with the compounds and then stained after 36 hours of treatment for the abundance of APO A1 using a commercially available human APO A1 antibody.

### EXAMPLE 68

### Measurement of ApoA-1 Promoter Induction

CaCo2 or Hep G2 cells are exposed to varying concentrations of the compounds. The cells are transfected, using a standard technique, with the reporter construct, pA1.474-Luc along with pRSV-β-galactosidase as a monitor for transfection efficiency. The pA1.474-Luc is a construct that was created using conventional molecular biology techniques and contains the human APO A1 promoter from −474 to −7 fused to the reporter, firefly luciferase (Luc) (U.S. patent application Ser. No. 10/222,013). Compounds are dissolved in DMSO and then added to the culture media for 16 hours. At the end of the treatment, the cells are harvested and the Luc-activity measured. Values are normalized to both lysate protein concentration and also β-galactosidase activity. Spent media exposed to the cells for 36 hours may be assayed for its content of APO A1 protein using western blot analysis.

### EXAMPLE 69

### Measurement of AGCCCCCGC Element Induction

CaCo2 or Hep G2 cells are exposed to varying concentrations of the compounds. The cells are transfected, using a standard technique, with a reporter construct, comprising the AGCCCCCGC element, operably linked to a promoter (for example the thymidine kinase (TK) promoter), operably linked to a reporter gene (for example luciferase, CAT, or apolipoprotein A1 itself), along with pRSV-β-galactosidase as a monitor for transfection efficiency as taught in U.S. patent application Ser. No. 10/222,013. Compounds are dissolved in DMSO and then added to the culture media for 16 hours. At the end of the treatment, the cells are harvested and the reporter gene activity measured. Values are normalized to both lysate protein concentration and also β-galactosidase activity.

### EXAMPLE 70

### Treatment of Fertility Conditions Using egr-1 Effectors

Egr-1 is known from knockout mouse experiments to be required for sufficient expression of leuteinizing hormone-beta, and the absence of egr-1 leads to the loss of reproductive capability in homozygous knockout mice. Modulation of activity mediated through egr-1 consensus sequence elements therefore represents a potential mechanism for treatment of humans or mammals to suppress fertility or conversely to enhance it, in individuals of reduced fertility.

### EXAMPLE 71

### Treatment of Cancer Using egr-1 Effectors

Egr-1 suppresses transformation by trans-activating transforming growth factor-beta (TGF-β). TGF-β is itself suppressed by a variety of cancers and modulation of activity mediated through egr-1 consensus sequence elements therefore represents a potential mechanism for treatment of cancer and other proliferative diseases in humans or mammals.

### EXAMPLE 72

### Treatment of Cancer Using egr-1 Effectors Acting on p21

Egr-1 cooperates with p21 (also known as CIP1 and Waft) to suppress transformation. This represents an alternate pathway by which egr-1 is involved in cancer and other proliferative diseases and therefore modulation of activity mediated through egr-1 consensus sequence elements represents a potential mechanism for the treatment of cancer or similar proliferative diseases in humans or mammals.

### EXAMPLE 73

### Treatment of Cancer Using egr-1 Effector Acting on p53

Egr-1 induces cell cycle arrest or apoptosis, depending on the severity of cellular injury, through trans-activating p53. Modulation of activity mediated through egr-1 consensus sequence elements therefore represents a potential mechanism for treatment of humans or mammals for disorders to which changes in p53 activation levels are associated, for example cancer. In some eases, cell cycle induced arrest may allow injured cells to respond to the injury and effect repair, representing another potential mechanism of action for treatments effected by the modulation of activity mediated through egr-1 consensus sequence elements.

### EXAMPLE 74

### Treatment of Prostrate Cancer Using egr-1 Effectors

Egr-1 is over-expressed in prostate tumor cancer cells, where it has been linked functionally to maintenance of the cancerous state. Modulation of activity mediated through egr-1 consensus sequence elements therefore represents a potential mechanism for the treatment of prostate cancer.

### EXAMPLE 75

### Treatment of Vascular Diseases Using egr-1 Effectors

Egr-1 increases activity levels of FGF-2, which in turn increases angiogenesis and stenosis, Modulating activity that is mediated through egr-1 consensus sequence elements therefore represents a potential therapeutic approach to down regulate angiogensis as a treatment for cancer. Alternatively, modulating activity that is mediated through egr-1 consensus sequence elements represents a potential therapeutic approach to down regulate the stenosis associated with numerous vascular diseases, including atherosclerosis, cerebrovascular disorders, and restenosis following angioplasty. Conversely, modulating activity that is mediated through egr-1 consensus sequence elements may represent a potential therapeutic approach to up-regulate angiogenesis to treat ischemie tissues, such as for wound healing therapeutic intervention.

### EXAMPLE 76

### Treatment of Inflammation and Pulmonary Disorders Using egr-1 Effectors

Egr-1 activation contributes to the sustained expression of inflammatory mediators, such as occurs in pulmonary disorders including emphysema and asthma. Modulating activity that is mediated through egr-1 consensus sequence elements therefore represents a potential therapeutic approach for the treatment of pulmonary disorders, such as emphysema, asthma, cystic fibrosis and chronic obstructive pulmonary disorder.

What is claimed is:

1. The use of a compound capable of modulating transcription arising from an egr-1 response element consensus sequence and expression state of a gene in manufacture of a medicament for the treatment of a disease or health condition associated with an expression state of a gene associated with an egr-1 response element consensus sequence.

2. The use of claim 1 wherein said compound comprises a compound selected from the group consisting of resveratrol, 3, 4′, 5 trinitroxy trans stilbene and 3, 4′, 5 tri(nitroxy)ethoxy trans stilbene, an analogue of any of the foregoing, and a pharmaceutically acceptable salt of any of the foregoing.

3. The use of claim 1 wherein said disease is selected from the group consisting of cancer and other proliferative diseases, vascular diseases, wounds requiring therapeutic intervention, inflammation, and pulmonary disorders.

4. The use of claim 3 wherein said pulmonary disorder is selected from emphysema, asthma, cystic fibrosis, chronic obstructive pulmonary disorder, CVD, atherosclerosis, hypertension and/or restenosis.

5. The use of claim 3 wherein said cancer related disorder is selected from the group consisting of cell cycle arrest or apoptosis disorders associated with altered p53 levels, and angiogenesis and stenosis associated with altered activity levels of FGF-2.

6. The use of claim 1 wherein said health condition is selected from the group consisting of fertility and infertility, vascular diseases, wounds requiring therapeutic intervention, inflammation, and pulmonary disorders.

7. The use of claim 6 wherein said vascular disease comprises atherosclerosis, cerebrovascular disorders, restenosis following angioplasty or ischemia.

8. The use of claim 1 wherein said egr-1 response element consensus sequence is associated with trans-activating transforming growth factor-beta (TGF-β).

9. The use of claim wherein said disease is selected from the group consisting of cancer and other proliferative diseases.

10. The use of claim 1 wherein said egr-1 response element consensus sequence is associated with leutenizing hormone.

11. The use of claim 10 wherein said health condition is reduced fertility.

12. The use of a compound capable of modulating transcription arising from an egr-1 response element consensus sequence and expression state of p21 in manufacture of a medicament for the treatment of a disease or health condition selected from the group consisting of cancer, other proliferative diseases, and susceptibility to cellular transformation.

13. The use of a compound capable of modulating transcription arising from an egr-1 response element consensus sequence and expression state of p53 in manufacture of a medicament for the treatment of a health condition requiring treatment selected from the group consisting of induced cell cycle arrest, cell injury and need for cell repair.

14. The use of a compound capable of modulating transcription arising from an egr-1 response element consensus sequence and expression state of FGF-2 in manufacture of a medicament for the treatment of a health condition requiring treatment selected from the group consist of angiogenesis and stenosis.

15. The use of claim 1 wherein said compound comprises resveratrol, 3, 4′, 5 trinitroxy trans stilbene and 3, 4′, 5 tri(nitroxy)ethoxy trans stilbene or an analogue thereof.

16. A method for identifying a compound capable of modulating expression of a gene associated with an egr-1 response element consensus sequence comprising providing an expression system comprising cells or cellular extracts and an egr-1 response element operably linked to a promoter and a gene whose expression can be modulated and measured, and determining whether said compound can induce modulation of expression in said expression system.

17. The method of claim 16 wherein said egr-1 response element consensus sequence comprises AGCCCCCGC.

18. The use of a compound identified by the method of claim 17 in manufacture of a medicament for the treatment of a disease or health condition.

19. The use of claim 18 wherein said compound comprises a compound with a donatable nitric oxide component and a free radical scavenging anti-oxidant molecule.

20. The use of claim 19 wherein said compound comprises resveratrol and analogues thereof comprising at least one nitric oxide donating moieties substituted for at least one naturally occurring hydroxyl group of said resveratrol.

21. The use of claim 20 wherein the compound is selected from the group consisting of 3, 4′, 5 trinitroxy trans stilbene and 3, 4′, 5 tri(nitroxy)ethoxy trans stilbene.

22. The use of claim 20 wherein said analogue is selected from the group of OCxNO2 substituted compounds.

23. The use of claim 22 wherein said analogue is a diazeniumdiolate analogue.

24. The use of claim 20 wherein at least one naturally occurring hydroxyl group of said resveratrol is substituted with sulphur or nitrogen.

25. A method for identifying a compound capable of modulating transcription arising from an egr-1 or an egr-1 consensus sequence element comprising the step of providing a test system comprising and egr-1 or an egr-1 consensus sequence element operably linked to a gene capable of expressing a detectable product, measuring a reference level of detectable product, contacting said test system with a compound to be tested and thereafter measuring the level of detectable product; comparing said detected level against the reference level and determining therefrom whether said compound is an effector of egr-1 or an egr-1 consensus sequence element.

26. A compound capable of modulating expression of a gene associated with an egr-1 response element consensus sequence comprising a donatable nitric oxide component and a free radical scavenging anti-oxidant molecule.

27. The compound of claim 26 comprising a flavonoid compound comprising the structure: 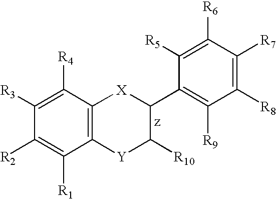
wherein
R1, R2, R3, R4, R5, R6, R7, R8, R9, R10, R13 and R14 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 or R13 or R14 is nitrooxy, R12, OR12, or OCOR12; and
Wherein
OCOR means 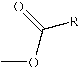
and R is R11 or R12
wherein
R11 is C₁₋₁₈, aryl, heteroaryl or a derivative thereof, wherein said derivative is optionally substituted and optionally branched, and may have one or more of the C atoms replaced by S, N or O, and
wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2; and
wherein
X can be O, CR13 or NR13;
Y can be CO [a ketone still maintaining the 6 atom ring structure], CR14 or NR14; and
Z can be a single or a double bond.

28. A pharmaceutical composition comprising the flavonoid compound of claim 27 in combination with a pharmaceutically acceptable carrier.

29. The use of a flavonoid compound according to claim 28 in manufacture of a medicament for the treatment of a disease or health condition associated with an expression state of a gene associated with an egr-1 response element consensus sequence.

30. The use of claim 29 wherein said disease is selected from the group consisting of cancer and other proliferative diseases, vascular diseases, wounds requiring therapeutic intervention, inflammation, and pulmonary disorders.

31. The use of claim 30 wherein said pulmonary disorder is selected from emphysema, asthma, cystic fibrosis, chronic obstructive pulmonary disorder, CVD, atherosclerosis, hypertension and/or restenosis.

32. The use of claim 30 wherein said cancer related disorder is selected from the group consisting of cell cycle arrest or apoptosis disorders associated with altered p53 levels, and anglogenesis and stenosis associated with altered activity levels of FGF-2.

33. The compound of claim 26 comprising an isoflavonoid compound comprising the structure: 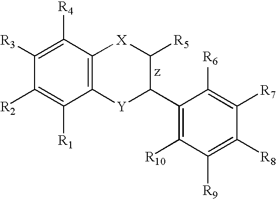
wherein
R1, R2, R3, P4, R5, R6, R7, R5, R9, R10, R13 and R14 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 or R13 or R14 is nitrooxy, R12, OR12, or OCOR12; and
wherein
OCOR means 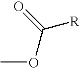
and R is R11 or R12
wherein
R11is C₁₋₁₈, aryl, heteroaryl or a derivative thereof, wherein said derivative is optionally substituted and optionally branched, and may have one or more of the C atoms replaced by S, N or O, and
wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2; and
wherein
X can be O, CR13 or NR13;
Y can be CO [a ketone still maintaining the 6 atom ring structure], CR14 or NR14; and
Z can be a single or a double bond.

34. A pharmaceutical composition comprising the isoflavonoid compound of claim 33 in combination with a pharmaceutically acceptable carrier.

35. The use of an isoflavonoid compound according to claim 34 in manufacture of a medicament for the treatment of a disease or health condition associated with an expression state of a gene associated with an egr-1 response element consensus sequence.

36. The use of claim 35 wherein said disease is selected from the group consisting of cancer and other proliferative diseases, vascular diseases, wounds requiring therapeutic intervention, inflammation, and pulmonary disorders.

37. The use of claim 36 wherein said pulmonary disorder is selected from emphysema, asthma, cystic fibrosis, chronic obstructive pulmonary disorder, CVD, atherosclerosis, hypertension and/or restenosis.

38. The use of claim 36 wherein said cancer related disorder is selected from the group consisting of cell cycle arrest or apoptosis disorders associated with altered p53 levels, and angiogenesis and stenosis associated with altered activity levels of FGF-2.

39. The compound of claim 26 comprising a stilbene compound comprising the following structure: 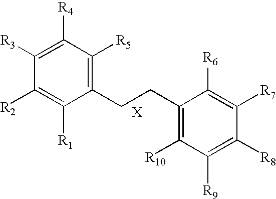
wherein
R1, R2, R3, R4, R5, R6, R7, R8, R9 and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
wherein
OCOR means 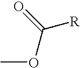
and R is R11 or R12
wherein
R11 is C₁₋₁₈, aryl, heteroaryl or a derivative thereof, wherein said derivative is optionally substituted and optionally branched, and may have one or more of the C atoms replaced by S, N or O, and
wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2 and
wherein
X can be a single, double or triple bond.

40. A pharmaceutical composition comprising the a stilbene compound of claim 39 in combination with a pharmaceutically acceptable carrier.

41. The use of a stilbene compound according to claim 40 in manufacture of a medicament for the treatment of a disease or health condition associated with an expression state of a gene associated with an egr-1 response element consensus sequence.

42. The use of claim 41 wherein said disease is selected from the group consisting of cancer and other proliferative diseases, vascular diseases, wounds requiring therapeutic intervention, inflammation, and pulmonary disorders.

43. The use of claim 42 wherein said pulmonary disorder is selected from emphysema, asthma, cystic fibrosis, chronic obstructive pulmonary disorder, CVD, atherosclerosis, hypertension and/or restenosis.

44. The use of claim 42 wherein said cancer related disorder is selected from the group consisting of cell cycle arrest or apoptosis disorders associated with altered p53 levels, and angiogenesis and stenosis associated with altered activity levels of FGF-2.

45. The compound of claim 26 comprising a chalcone compound comprising the following structure: 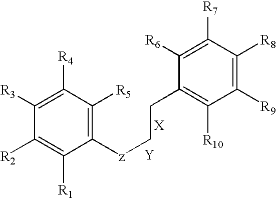
wherein
R1, R2, R3, R4, R5, R6, R7, R8, R9, R10, R13 and R14 may each be independently hydrogen, hydroxyl [OH], hydroxyalkyl aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-10 or R13 or R14 is nitrooxy, R12, OR12, or OCOR12; and
wherein
OCOR means 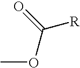
and R is R11 or R12
wherein
R11 is C₁₋₁₈, aryl, heteroaryl or a derivative thereof, wherein said derivative is optionally substituted and optionally branched, and may have one or more of the C atoms replaced by S, N or O, and
wherein
R12 is C₁₋₁₈, aryl, heteroaryl or a derivative thereof, wherein said derivative is optionally substituted, optionally branched, may have one or more of the C atoms replaced by S, N or O, and optionally containing one or more ONO.sub.2;
wherein
X can be a single or a double bond,
Y can be a single or a double bond
Z can be CO [a ketone] CR13 or NR13.

46. A pharmaceutical composition comprising the a chalcone compound of claim 45 in combination with a pharmaceutically acceptable carrier.

47. The use of a chalcone compound according to claim 46 in manufacture of a medicament for the treatment of a disease or health condition associated with an expression state of a gene associated with an egr-1 response element consensus sequence.

48. The use of claim 47 wherein said disease is selected from the group consisting of cancer and other proliferative diseases, vascular diseases, wounds requiring therapeutic intervention, inflammation, and pulmonary disorders.

49. The use of claim 48 wherein said pulmonary disorder is selected from emphysema, asthma, cystic fibrosis, chronic obstructive pulmonary disorder, CVD, atherosclerosis, hypertension and/or restenosis.

50. The use of claim 48 wherein said cancer related disorder is selected from the group consisting of cell cycle arrest or apoptosis disorders associated with altered p53 levels, and angiogenesis and stenosis associated with altered activity levels of FGF-2.

51. The compound of claim 26 comprising a polyphenol compound comprising the following structure: 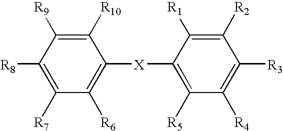
wherein
R1, R2, R3, R4, R5, R6, R7, R8, R9, and R10 may each be independently hydrogen, hydroxyl [OH], hydroxyalkyl, aminoalkyl, Bromide (Br), Iodide (I), nitrooxy [ONO.sub.2], methoxy [OCH.sub.3], ethoxy [OCH.sub2CH.sub.3], fluoride [F], chloride [Cl], CF.sub.3, CCl.sub.3, phosphate, R11, R12, OR11, OR12, OCOR11, OCOR12, O-sulfate [the sulfate conjugate], or O-glucoronidate [the glucoronic (AKA glucuronic) acid conjugates], with the proviso that at least one of R1-R10 is nitrooxy, R12, OR12, or OCOR12; and
wherein
OCOR means 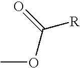
and R is R 11 or R12
wherein
R11 is C1-18, aryl, heteroaryl or a derivative thereof, wherein said derivative is optionally substituted and optionally branched, and may have one or more of the C atoms replaced by S, N or O, and
wherein
R12 is C1-18, aryl, heteroaryl or a derivative thereof, wherein said derivative is optionally substituted, optionally branched, may have one or more of the C atoms replaced by S, N or O, and optionally containing one or more ONO.sub.2; and
wherein
X can be C, S, (CO), SO, AKA ketone, (SO.sub.2)N, (CO)C, (CO)N, (CO)O, C—N [single bond], C═N [double bond], C—O, N—O, N—N [single bond], or N═N [double bond].

52. A pharmaceutical composition comprising the a polyphenol compound of claim 51 in combination with a pharmaceutically acceptable carrier.

53. The use of a polyphenol compound according to claim 52 in manufacture of a medicament for the treatment of a disease or health condition associated with an expression state of a gene associated with an egr-1 response element consensus sequence.

54. The use of claim 53 wherein said disease is selected from the group consisting of cancer and other proliferative diseases, vascular diseases, wounds requiring therapeutic intervention, inflammation, and pulmonary disorders.

55. The use of claim 54 wherein said pulmonary disorder is selected from emphysema, asthma, cystic fibrosis, chronic obstructive pulmonary disorder, CVD, atherosclerosis, hypertension and/or restenosis.

56. The use of claim 54 wherein said cancer related disorder is selected from the group consisting of cell cycle arrest or apoptosis disorders associated with altered p53 levels, and angiogenesis and stenosis associated with altered activity levels of FGF-2.


SEQUENCE LISTING (3 sequences)

SEQ ID NO: 1 - DNA, 21 bp, Rattus sp.
  APO AI Site “S”
tgcagcccccgcagcttcctg

SEQ ID NO: 2 - DNA, 21 bp, Homo sapiens
  APO AI Site “S”
tgcagcccccgcagcttgctg

SEQ ID NO: 3 - DNA, 9 bp, Artificial Sequence
  Description of Artificial Sequence EGR-1 Response Element Consensus Sequence
agcccccgc